SEQUENCE LISTING (20 sequences)

SEQ ID NO: 1 - protein, 350 aa, Artificial Sequence
  GPCR C5AR
MNSFNYTTPDYGHYDDKDTLDLNTPVDKTSNTLRVPDILALVIFAVVFLVGVLGNALVVWVTAFEAKRTINAIWFLNLAVADFLSCLALPILFTSIVQHHHWPFGGAACSILPSLILLNMYASILLLATISADRFLLVFKPIWCQNFRGAGLAWIACAVAWGLALLLTIPSFLYRVVREEYFPPKVLCGVDYSHDKRRERAVAIVRLVLGFLWPLLTLTICYTFILLRTWSRRATRSTKTLKVVVAVVASFFIFWLPYQVTGIMMSFLEPSSPTFLLLNKLDSLCVSFAYINCCINPIIYVVAGQGFQGRLRKSLPSLLRNVLTEESVVRESKSFTRSTVDTMAQKTQAV

SEQ ID NO: 2 - DNA, 1053 bp, Artificial Sequence
  GPCR C5AR
atgaactccttcaattataccacccctgattatgggcactatgatgacaaggataccctggacctcaacacccctgtggataaaacttctaacacgctgcgtgttccagacatcctggccttggtcatctttgcagtcgtcttcctggtgggagtgctgggcaatgccctggtggtctgggtgacggcattcgaggccaagcggaccatcaatgccatctggttcctcaacttggcggtagccgacttcctctcctgcctggcgctgcccatcttgttcacgtccattgtacagcatcaccactggccctttggcggggccgcctgcagcatcctgccctccctcatcctgctcaacatgtacgccagcatcctgctcctggccaccatcagcgccgaccgctttctgctggtgtttaaacccatctggtgccagaacttccgaggggccggcttggcctggatcgcctgtgccgtggcttggggtttagccctgctgctgaccataccctccttcctgtaccgggtggtccgggaggagtactttccaccaaaggtgttgtgtggcgtggactacagccacgacaaacggcgggagcgagccgtggccatcgtccggctggtcctgggcttcctgtggcctctactcacgctcacgatttgttacactttcatcctgctccggacgtggagccgcagggccacgcggtccaccaagacactcaaggtggtggtggcagtggtggccagtttctttatcttctggttgccctaccaggtgacggggataatgatgtccttcctggagccatcgtcacccaccttcctgctgctgaataagctggactccctgtgtgtctcctttgcctacatcaactgctgcatcaaccccatcatctacgtggtggccggccagggcttccagggccgactgcggaaatccctccccagcctcctccggaacgtgttgactgaagagtccgtggttagggagagcaagtcattcacgcgctccacagtggacactatggcccagaagacccaggcagtgtag

SEQ ID NO: 3 - protein, 426 aa, Artificial Sequence
  GPCR NMUR1
MTPLCLNCSVLPGDLYPGGARNPMACNGSAARGHFDPEDLNLTDEALRLKYLGPQQTELFMPICATYLLIFVVGAVGNGLTCLVILRHKAMRTPTNYYLFSLAVSDLLVLLVGLPLELYEMWHNYPFLLGVGGCYFRTLLFEMVCLASVLNVTALSVERYVAVVHPLQARSMVTRAHVRRVLGAVWGLAMLCSLPNTSLHGIQQLHVPCRGPVPDSAVCMLVRPRALYNMVVQTTALLFFCLPMAIMSVLYLLIGLRLRRERLLLMQEAKGRGSAAARSRYTCRLQQHDRGRRQVTKMLFVLVVVFGICWAPFHADRVMWSVVSQWTDGLHLAFQHVHVISGIFFYLGSAANPVLYSLMSSRFRETFQEALCLGACCHRLRPRHSSHSLSRMTTGSTLCDVGSLGSWVHPLAGNDGPEAQQETDPS

SEQ ID NO: 4 - DNA, 1281 bp, Artificial Sequence
  GPCR NMUR1
atgactcctctctgcctcaattgctctgtcctccctggagacctgtacccagggggtgcaaggaaccccatggcttgcaatggcagtgcggccagggggcactttgaccctgaggacttgaacctgactgacgaggcactgagactcaagtacctggggccccagcagacagagctgttcatgcccatctgtgccacatacctgctgatcttcgtggtgggcgctgtgggcaatgggctgacctgtctggtcatcctgcgccacaaggccatgcgcacgcctaccaactactacctcttcagcctggccgtgtcggacctgctggtgctgctggtgggcctgcccctggagctctatgagatgtggcacaactaccccttcctgctgggcgttggtggctgctatttccgcacgctactgtttgagatggtctgcctggcctcagtgctcaacgtcactgccctgagcgtggaacgctatgtggccgtggtgcacccactccaggccaggtccatggtgacgcgggcccatgtgcgccgagtgcttggggccgtctggggtcttgccatgctctgctccctgcccaacaccagcctgcacggcatccagcagctgcacgtgccctgccggggcccagtgccagactcagctgtttgcatgctggtccgcccacgggccctctacaacatggtagtgcagaccaccgcgctgctcttcttctgcctgcccatggccatcatgagcgtgctctacctgctcattgggctgcgactgcggcgggagaggctgctgctcatgcaggaggccaagggcaggggctctgcagcagccaggtccagatacacctgcaggctccagcagcacgatcggggccggagacaagtgaccaagatgctgtttgtcctggtcgtggtgtttggcatctgctgggccccgttccacgccgaccgcgtcatgtggagcgtcgtgtcacagtggacagatggcctgcacctggccttccagcacgtgcacgtcatctccggcatcttcttctacctgggctcggcggccaaccccgtgctctatagcctcatgtccagccgcttccgagagaccttccaggaggccctgtgcctcggggcctgctgccatcgcctcagaccccgccacagctcccacagcctcagcaggatgaccacaggcagcaccctgtgtgatgtgggctccctgggcagctgggtccaccccctggctgggaacgatggcccagaggcgcagcaagagaccgatccatcctga

SEQ ID NO: 5 - protein, 377 aa, Artificial Sequence
  GPCR P2RY2
MAADLGPWNDTINGTWDGDELGYRCRFNEDFKYVLLPVSYGVVCVPGLCLNAVALYIFLCRLKTWNASTTYMFHLAVSDALYAASLPLLVYYYARGDHWPFSTVLCKLVRFLFYTNLYCSILFLTCISVHRCLGVLRPLRSLRWGRARYARRVAGAVWVLVLACQAPVLYFVTTSARGGRVTCHDTSAPELFSRFVAYSSVMLGLLFAVPFAVILVCYVLMARRLLKPAYGTSGGLPRAKRKSVRTIAVVLAVFALCFLPFHVTRTLYYSFRSLDLSCHTLNAINMAYKVTRPLASANSCLDPVLYFLAGQRLVRFARDAKPPTGPSPATPARRRLGLRRSDRTDMQRIEDVLGSSEDSRRTESTPAGSENTKDIRL

SEQ ID NO: 6 - DNA, 1134 bp, Artificial Sequence
  GPCR P2RY2
atggcagcagacctgggcccctggaatgacaccatcaatggcacctgggatggggatgagctgggctacaggtgccgcttcaacgaggacttcaagtacgtgctgctgcctgtgtcctacggcgtggtgtgcgtgcctgggctgtgtctgaacgccgtggcgctctacatcttcttgtgccgcctcaagacctggaatgcgtccaccacatatatgttccacctggctgtgtctgatgcactgtatgcggcctccctgccgctgctggtctattactacgcccgcggcgaccactggcccttcagcacggtgctctgcaagctggtgcgcttcctcttctacaccaacctttactgcagcatcctcttcctcacctgcatcagcgtgcaccggtgtctgggcgtcttacgacctctgcgctccctgcgctggggccgggcccgctacgctcgccgggtggccggggccgtgtgggtgttggtgctggcctgccaggcccccgtgctctactttgtcaccaccagcgcgcgcgggggccgcgtaacctgccacgacacctcggcacccgagctcttcagccgcttcgtggcctacagctcagtcatgctgggcctgctcttcgcggtgccctttgccgtcatccttgtctgttacgtgctcatggctcggcgactgctaaagccagcctacgggacctcgggcggcctgcctagggccaagcgcaagtccgtgcgcaccatcgccgtggtgctggctgtcttcgccctctgcttcctgccattccacgtcacccgcaccctctactactccttccgctcgctggacctcagctgccacaccctcaacgccatcaacatggcctacaaggttacccggccgctggccagtgctaacagttgccttgaccccgtgctctacttcctggctgggcagaggctcgtacgctttgcccgagatgccaagccacccactggccccagccctgccaccccggctcgccgcaggctgggcctgcgcagatccgacagaactgacatgcagaggatagaagatgtgttgggcagcagtgaggactctaggcggacagagtccacgccggctggtagcgagaacactaaggacattcggctgtag

SEQ ID NO: 7 - protein, 342 aa, Artificial Sequence
  GPCR PTAFR
MEPHDSSHMDSEFRYTLFPIVYSIIFVLGVIANGYVLWVFARLYPCKKFNEIKIFMVNLTMADMLFLITLPLWIVYYQNQGNWILPKFLCNVAGCLFFINTYCSVAFLGVITYNRFQAVTRPIKTAQANTRKRGISLSLVIWVAIVGAASYFLILDSTNTVPDSAGSGNVTRCFEHYEKGSVPVLIIHIFIVFSFFLVFLIILFCNLVIIRTLLMQPVQQQRNAEVKRRALWMVCTVLAVFIICFVPHHVVQLPWTLAELGFQDSKFHQAINDAHQVTLCLLSTNCVLDPVIYCFLTKKFRKHLTEKFYSMRSSRKCSRATTDTVTEVVVPFNQIPGNSLKN

SEQ ID NO: 8 - DNA, 1029 bp, Artificial Sequence
  GPCR PTAFR
atggagccacatgactcctcccacatggactctgagttccgatacactctcttcccgattgtttacagcatcatctttgtgctcggggtcattgctaatggctacgtgctgtgggtctttgcccgcctgtacccttgcaagaaattcaatgagataaagatcttcatggtgaacctcaccatggcggacatgctcttcttgatcaccctgccactttggattgtctactaccaaaaccagggcaactggatactccccaaattcctgtgcaacgtggctggctgccttttcttcatcaacacctactgctctgtggccttcctgggcgtcatcacttataaccgcttccaggcagtaactcggcccatcaagactgctcaggccaacacccgcaagcgtggcatctctttgtccttggtcatctgggtggccattgtgggagctgcatcctacttcctcatcctggactccaccaacacagtgcccgacagtgctggctcaggcaacgtcactcgctgctttgagcattacgagaagggcagcgtgccagtcctcatcatccacatcttcatcgtgttcagcttcttcctggtcttcctcatcatcctcttctgcaacctggtcatcatccgtaccttgctcatgcagccggtgcagcagcagcgcaacgctgaagtcaagcgccgggcgctgtggatggtgtgcacggtcttggcggtgttcatcatctgcttcgtgccccaccacgtggtgcagctgccctggacccttgctgagctgggcttccaggacagcaaattccaccaggccattaatgatgcacatcaggtcaccctctgcctccttagcaccaactgtgtcttagaccctgttatctactgtttcctcaccaagaagttccgcaagcacctcaccgaaaagttctacagcatgcgcagtagccggaaatgctcccgggccaccacggatacggtcactgaagtggttgtgccattcaaccagatccctggcaattccctcaaaaattag

SEQ ID NO: 9 - protein, 380 aa, Artificial Sequence
  GPCR AGTRL1
MEEGGDFDNYYGADNQSECEYTDWKSSGALIPAIYMLVFLLGTTGNGLVLWTVFRSSREKRRSADIFIASLAVADLTFVVTLPLWATYTYRDYDWPFGTFFCKLSSYLIFVNMYASVFCLTGLSFDRYLAIVRPVANARLRLRVSGAVATAVLWVLAALLAMPVMVLRTTGDLENTTKVQCYMDYSMVATVSSEWAWEVGLGVSSTTVGFVVPFTIMLTCYFFIAQTIAGHFRKERIEGLRKRRRLLSIIVVLVVTFALCWMPYHLVKTLYMLGSLLHWPCDFDLFLMNIFPYCTCISYVNSCLNPFLYAFFDPRFRQACTSMLCCGQSRCAGTSHSSSGEKSASYSSGHSQGPGPNMGKGGEQMHEKSIPYSQETLVVD

SEQ ID NO: 10 - DNA, 1143 bp, Artificial Sequence
  GPCR AGTRL1
atggaggaaggtggtgattttgacaactactatggggcagacaaccagtctgagtgtgagtacacagactggaaatcctcgggggccctcatccctgccatctacatgttggtcttcctcctgggcaccacgggcaacggtctggtgctctggaccgtgtttcggagcagccgggagaagaggcgctcagctgatatcttcattgctagcctggcggtggctgacctgaccttcgtggtgacgctgcccctgtgggctacctacacgtaccgggactatgactggccctttgggaccttcttctgcaagctcagcagctacctcatcttcgtcaacatgtacgccagcgtcttctgcctcaccggcctcagcttcgaccgctacctggccatcgtgaggccagtggccaatgctcggctgaggctgcgggtcagcggggccgtggccacggcagttctttgggtgctggccgccctcctggccatgcctgtcatggtgttacgcaccaccggggacttggagaacaccactaaggtgcagtgctacatggactactccatggtggccactgtgagctcagagtgggcctgggaggtgggccttggggtctcgtccaccaccgtgggctttgtggtgcccttcaccatcatgctgacctgttacttcttcatcgcccaaaccatcgctggccacttccgcaaggaacgcatcgagggcctgcggaagcggcgccggctgctcagcatcatcgtggtgctggtggtgacctttgccctgtgctggatgccctaccacctggtgaagacgctgtacatgctgggcagcctgctgcactggccctgtgactttgacctcttcctcatgaacatcttcccctactgcacctgcatcagctacgtcaacagctgcctcaaccccttcctctatgcctttttcgacccccgcttccgccaggcctgcacctccatgctctgctgtggccagagcaggtgcgcaggcacctcccacagcagcagtggggagaagtcagccagctactcttcggggcacagccaggggcccggccccaacatgggcaagggtggagaacagatgcacgagaaatccatcccctacagccaggagacccttgtggttgactag

SEQ ID NO: 11 - protein, 482 aa, Artificial Sequence
  GPCR C3AR
MASFSAETNSTDLLSQPWNEPPVILSMVILSLTFLLGLPGNGLVLWVAGLKMQRTVNTIWFLHLTLADLLCCLSLPFSLAHLALQGQWPYGRFLCKLIPSIIVLNMFASVFLLTAISLDRCLVVFKPIWCQNHRNVGMACSICGCIWVVAFVMCIPVFVYREIFTTDNHNRCGYKFGLSSSLDYPDFYGDPLENRSLENIVQPPGEMNDRLDPSSFQTNDHPWTVPTVFQPQTFQRPSADSLPRGSARLTSQNLYSNVFKPADVVSPKIPSGFPIEDHETSPLDNSDAFLSTHLKLFPSASSNSFYESELPQGFQDYYNLGQFTDDDQVPTPLVAITITRLVVGFLLPSVIMIACYSFIVFRMQRGRFAKSQSKTFRVAVVVVAVFLVCWTPYHIFGVLSLLTDPETPLGKTLMSWDHVCIALASANSCFNPFLYALLGKDFRKKARQSIQGILEAAFSEELTRSTHCPSNNVISERNSTTV

SEQ ID NO: 12 - DNA, 1449 bp, Artificial Sequence
  GPCR C3AR
atggcgtctttctctgctgagaccaattcaactgacctactctcacagccatggaatgagcccccagtaattctctccatggtcattctcagccttacttttttactgggattgccaggcaatgggctggtgctgtgggtggctggcctgaagatgcagcggacagtgaacacaatttggttcctccacctcaccttggcggacctcctctgctgcctctccttgcccttctcgctggctcacttggctctccagggacagtggccctacggcaggttcctatgcaagctcatcccctccatcattgtcctcaacatgtttgccagtgtcttcctgcttactgccattagcctggatcgctgtcttgtggtattcaagccaatctggtgtcagaatcatcgcaatgtagggatggcctgctctatctgtggatgtatctgggtggtggcttttgtgatgtgcattcctgtgttcgtgtaccgggaaatcttcactacagacaaccataatagatgtggctacaaatttggtctctccagctcattagattatccagacttttatggagatccactagaaaacaggtctcttgaaaacattgttcagccgcctggagaaatgaatgataggttagatccttcctctttccaaacaaatgatcatccttggacagtccccactgtcttccaacctcaaacatttcaaagaccttctgcagattcactccctaggggttctgctaggttaacaagtcaaaatctgtattctaatgtatttaaacctgctgatgtggtctcacctaaaatccccagtgggtttcctattgaagatcacgaaaccagcccactggataactctgatgcttttctctctactcatttaaagctgttccctagcgcttctagcaattccttctacgagtctgagctaccacaaggtttccaggattattacaatttaggccaattcacagatgacgatcaagtgccaacacccctcgtggcaataacgatcactaggctagtggtgggtttcctgctgccctctgttatcatgatagcctgttacagcttcattgtcttccgaatgcaaaggggccgcttcgccaagtctcagagcaaaacctttcgagtggccgtggtggtggtggctgtctttcttgtctgctggactccataccacatttttggagtcctgtcattgcttactgacccagaaactcccttggggaaaactctgatgtcctgggatcatgtatgcattgctctagcatctgccaatagttgctttaatcccttcctttatgccctcttggggaaagattttaggaagaaagcaaggcagtccattcagggaattctggaggcagccttcagtgaggagctcacacgttccacccactgtccctcaaacaatgtcatttcagaaagaaatagtacaactgtgtga

SEQ ID NO: 13 - protein, 412 aa, Artificial Sequence
  GPCR CCR5
MDYQVSSPIYDINYYTSEPCQKINVKQIAARLLPPLYSLVFIFGFVGNMLVILILINCKRLKSMTDIYLLNLAISDLFFLLTVPFWAHYAAAQWDFGNTMCQLLTGLYFIGFFSGIFFIILLTIDRYLAVVHAVFALKARTVTFGVVTSVITWVVAVFASLPGIIFTRSQKEGLHYTCSSHFPYSQYQFWKNFQTLKIVILGLVLPLLVMVICYSGILKTLLRCRNEKKRHRAVRLIFTIMIVYFLFWAPYNIVLLLNTFQEFFGLNNCSSSNRLDQAMQVTETLGMTHCCINPIIYAFVGEKFRNYLLVFFQKHIAKRFCKCCSIFQQEAPERASSVYTRSTGEQEISVGLATGGATTATCAAGTGTCAAGTCCAATCTATGACATCAATTATTATACATCGGAGCCCTGC

SEQ ID NO: 14 - DNA, 999 bp, Artificial Sequence
  GPCR CCR5
caaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtga

SEQ ID NO: 15 - protein, 352 aa, Artificial Sequence
  GPCR CXCR4
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 16 - DNA, 1059 bp, Artificial Sequence
  GPCR CXCR4
atggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaa

SEQ ID NO: 17 - protein, 397 aa, Artificial Sequence
  GPCR PAR2
MRSPSAAWLLGAAILLAASLSCSGTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 18 - DNA, 1194 bp, Artificial Sequence
  GPCR PAR2
atgcggagccccagcgcggcgtggctgctgggggccgccatcctgctagcagcctctctctcctgcagtggcaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattga

SEQ ID NO: 19 - DNA, 5796 bp, Artificial Sequence
  vector pMEX2
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagccaccatggagacagacacactcctgctatgggtactgctgctctgggttccaggttccactggtgatatcgactataaagatgatgacgacaagggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctgatagtcgacgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcataagtagtactctggagttcgaaatgaccgaccaagcgacgcccaacctgccatcacgatggccgcaataaaatatctttattttcattacatctgtgtgttggttttttgtgtgaatcgatagcgataaagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 20 - DNA, 5627 bp, Artificial Sequence
  vector pMEX5
tctagactgtatgtacatacagagttcttgagtgatccctgtatgtacatacaggtcatcatgaagtagtctgtatgtacatacagagaacttgagtgatccctgtatgtacatacagttcaagatacttagttctgtatgtacatacagagttcttgagtgatccctgtatgtacatacagtctagagttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggctagcctcgagccaccatggagacagacacactcctgctatgggtactgctgctctgggttccaggttccactggtgatatcgactataaagatgatgacgacaagggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctgatagtcgacgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcataagtagtactctggagttcgaaatgaccgaccaagcgacgcccaacctgccatcacgatggccgcaataaaatatctttattttcattacatctgtgtgttggttttttgtgtgaatcgatagcgataaagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

(19) United States
(12) Patent Application Publication
(10) Pub. No.: US 2012/0219968 A1
(43) Pub. Date: Aug. 30, 2012

(54) GPCR Expressing Cell Lines and Antibodies

(75) Inventors: Helena S. Mancebo, Fremont, CA (US); Jeng-Horng Her, San Jose, CA (US); Samuel X. Li, Redmond, WA (US); Jianfu L. Wang, Union City, CA (US)

(73) Assignee: Multispan, Inc., Hayward, CA (US)

(21) Appl. No.: 13/470,665

(22) Filed: May 14, 2012

Related U.S. Application Data
(63) Continuation of application No. 12/820,987, filed on Jun. 22, 2010, now Pat. No. 8,178,346.
Application No. 12/820,987 is a continuation of application No. 11/529,826, filed on Sep. 29, 2006, now Pat. No. 7,781,209.
(60) Provisional application No. 60/730,997, filed on Oct. 28, 2005.

(51) Int. Cl. G01N 33/566; G01N 21/64; G01N 33/577

(52) U.S. Cl. 435/7.21

(57) ABSTRACT

The present invention provides expression vectors that facilitate high levels of expression of GPCR proteins. Encompassed by the invention are methods and compositions for recombinant cell lines expressing GPCR proteins with the aid of the expression vectors of the instant invention. The recombinant cell lines of the instant invention express GPCR proteins at levels of at least about 150,000 copies of the protein per cell. The present invention also provides methods and compositions for raising antibodies against GPCR proteins using the high expressing recombinant cells of the instant invention.

### CROSS-REFERENCE TO RELATED APPLICATIONS

This application is a continuation of U.S. patent application Ser. No. 12/820,987 filed Jun. 22, 2010, now U.S. Pat. No. 8,178,346, which application is a continuation of U.S. patent application Ser. No. 11/529,826 filed Sep. 29, 2006, now U.S. Pat. No. 7,781,209, which application claims the benefit of U.S. Provisional Patent Applications No. 60/730,997, filed Oct. 28, 2005, all of which are incorporated by reference as if fully disclosed herein.

### SEQUENCE LISTING

A Sequence Listing in computer readable form (CRF) is submitted with this application. The CRF file is named 191332US03.5T25.txt, was created on Aug. 11, 2010, and contains 60 kilobytes. The entire contents of the CRF file are incorporated herein by this reference.

### BACKGROUND OF THE INVENTION

This invention relates generally to the field of G protein coupled receptor (GPCR) expression and modulation.

G protein-coupled receptors (GPCRs) are a historically successful therapeutic target family, with GPCR-directed drugs covering a wide range of therapeutic indications. As cell surface receptors, GPCRs are vital to cellular functioning, because they are primary mediators of cell to cell communication.

Mammalian cells express very low levels of endogenous GPCRs, generally with no more than three thousand copies per cell. This level is sufficient for receptor function, but offers a challenge to GPCR research, which often requires much higher concentrations of functional proteins. For example, structural studies, small molecule drug design and generation of functional antibodies against the native GPCR conformation require expression levels that are orders of magnitude higher than what is seen using current methods.

Attempts have been made to isolate mammalian cell lines that overexpress exogenous GPCRs, but these past attempts have failed due to the cellular toxicity that occurs with receptor overexpression. Attempts to create expression systems in “lower” organisms have similarly met with limited success due to inefficient folding (bacteria), low yield (yeast) or incorrect post-translation modification (baculovirus).

A need thus exists for stable, high-expression systems capable of providing multiple copies of GPCR proteins for structural and functional studies.

### SUMMARY OF THE INVENTION

Accordingly, the present invention provides a novel method for development of mammalian cell lines that overexpress G protein coupled receptor (GPCR) proteins. Exemplary cell lines of the invention express GPCR at levels upwards of one million copies per cell. Such high levels of expression are surprising, given that conventional methods of expression yield much lower levels of expression for transmembrane proteins.

In a first aspect, the present invention provides a vector for facilitating high levels of expression of GPCR proteins in a cell line. The vector includes components such as a cytomegalovirus (CMV) promoter, a signal peptide, and epitope tag, a Kozak sequence, a poly-A site, and a viral origin of replication.

In second aspect, the invention provides a recombinant cell line which expresses GPCR proteins at a level of at least 150,000 copies per cell. In a further aspect, the invention provides methods for producing recombinant cell lines by transfecting a host cell with at least one expression vector. In a preferred embodiment of the invention, the expression vector may include a nucleotide sequence selected from SEQ ID NO: 19 and SEQ ID NO: 20.

In a still further aspect, the invention provides methods for using recombinant cell lines to screen for therapeutic candidates able to interact with a GPCR protein. The method includes expressing a GPCR amino acid sequence in a recombinant cell. A test entity is contacted with a region of the GPCR amino acid sequence, and this region presents a fragment of the GPCR amino acid sequence that is sufficient for the test entity to interact with the fragment. In an embodiment of the invention, the test entity interacts with the fragment in a detectable manner. Detection of the interaction between the test entity and the fragment of the GPCR amino acid sequence identifies the test entity as a therapeutic candidate.

In a still further aspect, the invention provides a method of using a GPCR-expressing cell line to identify a test compound which modulates the activity of the GPCR protein. This method includes making a first measurement, which involves measuring second messenger activity in the cell line in the absence of the test compound, and making a second measurement, which involves measuring second messenger activity in the presence of the test compound. The method encompasses a comparison of the first and second measurement to determine if there is a difference between the two. A difference between the first and second measurement identifies the test compound as a compound that modulates the activity of the GPCR. In a preferred embodiment of the invention, the cell line expresses at least 150,000 copies of the GPCR protein per cell.

In another aspect, the invention provides an antibody or antigen binding fragment that is able to bind to a structural feature of a GPCR protein. In a further aspect of the invention, the antibody or antigen binding fragment is raised against an immunogen which is a cell line expressing between 150,000 and 2,000,000 copies of GPCR protein per cell.

In a further aspect of the invention, a method is provided whereby cells expressing GPCR proteins are detected in a test sample. The test sample is contacted with an antibody specifically binding to a structural feature of a GPCR protein. Specific binding of the antibody to a structural feature of a GPCR protein identifies the presence of GPCR-expressing cells in the test sample. This method further includes the detection of specific binding of the antibody to a structural feature of a GPCR protein.

In a still further aspect of the invention, a method is provided for producing monoclonal antibodies for a GPCR protein. In this aspect of the invention, a test animal is immunized with at least one cell line expressing a GPCR protein, and the cell line preferably expresses at least 50,000 copies of said GPCR protein per cell. The test animal is induced to produce hybridomas, and the method includes isolating the hybridomas and screening for monoclonal antibodies using one or more cell-based assay systems.

In another aspect, the invention provides a kit for high throughput purification and quantification of recombinant proteins of one or more members of one or more GPCR families. A kit according to the invention comprises a vector for expressing the recombinant proteins at levels between 50,000 and 2,000,000 copies per cell. A kit according to the invention can also comprise an affinity chromatography resin, a proteolytic enzyme, an internal quantification standard, a matrix for MALDI-TOF mass spectrometry, as well as instructions for use of the kit.

In still another aspect, the invention provides a method for identifying a DNA sequence encoding a member of a GPCR family. This method includes the process of probing a cDNA library or genomic library with a labeled probe and identifying from the library sequences able to hybridize to the probe under stringent conditions. Encompassed in the scope of the invention are labeled probes comprising nucleotide sequences selected from SEQ ID NO: 1, 3, 5, 7, 9, 11, 13, and 17.

In yet another aspect, the invention provides a method for producing a functional assay cell line. This method includes producing a cell line expressing a GPCR protein and coupling a functional reporter to binding of a ligand to the GPCR protein. The functional reporter is such that a binding event between the ligand and the GPCR protein is detectable as a reporter activity readout. In an exemplary embodiment of the invention, the cell line expresses a GPCR protein comprising an amino acid sequence selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, and SEQ ID NO: 17. In a preferred embodiment of the invention, the cell line expresses at least 150,000 copies of the GPCR protein per cell.

### BRIEF DESCRIPTION OF THE DRAWINGS

FIG. 1 displays the amino acid sequence (SEQ ID NO: 1) and the nucleotide sequence (SEQ ID NO: 2) for the G-protein coupled receptor CSAR.

FIG. 2 displays the amino acid sequence (SEQ ID NO: 3) and the nucleotide sequence (SEQ ID NO: 4) for the G-protein coupled receptor NMUR1.

FIG. 3 displays the amino acid sequence (SEQ ID NO: 5) and the nucleotide sequence (SEQ ID NO: 6) for the G-protein coupled receptor P2RY2.

FIG. 4 displays the amino acid sequence (SEQ ID NO: 7) and the nucleotide sequence (SEQ ID NO: 8) for the G-protein coupled receptor PTAFR.

FIG. 5 displays the amino acid sequence (SEQ ID NO: 9) and the nucleotide sequence (SEQ ID NO: 10) for the G-protein coupled receptor AGTRL1.

FIG. 6 displays the amino acid sequence (SEQ ID NO: 11) and the nucleotide sequence (SEQ ID NO: 12) for the G-protein coupled receptor C3AR.

FIG. 7 displays the amino acid sequence (SEQ ID NO: 13) and the nucleotide sequence (SEQ ID NO: 14) for the G-protein coupled receptor CCR5.

FIG. 8 displays the amino acid sequence (SEQ ID NO: 15) and the nucleotide sequence (SEQ ID NO: 16) for the G-protein coupled receptor CXCR4.

FIG. 9 displays the amino acid sequence (SEQ ID NO: 17) and the nucleotide sequence (SEQ ID NO: 18) for the G-protein coupled receptor PAR2.

FIG. 10 is a schematic map of the features of the GPCR expression vector pMEX2.

FIG. 11A and FIG. 11B show the nucleotide sequence of the GPCR expression vector pMEX2.

FIG. 12 is a schematic map of the features of the GPCR expression vector pMEX5.

FIG. 13A and FIG. 13B show the nucleotide sequence of the GPCR expression vector pMEX5.

FIG. 14 displays the results of an ELISA assay of mouse immune sera collected after 3 immunizations with CXCR4 transfected cells as the immunogen.

FIG. 15 displays data from FITC analysis of cell surface expression for the identified G-protein coupled receptors (GPCRs).

FIG. 16 provides a surface expression profile of 72 recombinant GPCRs as determined by FACS analysis.

FIG. 17 provides data from a binding assay of transiently expressed histamine receptors (H2).

FIG. 18 provides data from a cell surface assay for the identified GPCRs transfected into HEK293T cells.

FIG. 19 displays data from a calcium signaling assay for the GPCRs EDG4 (CHO cells) and NMUR1 (HEK293T cells). CHO/Flag-EDG4 is a stable cell line used as an immunogen. The traces in the top row are from negative antiserum tested by Flag peptide ELISA. The traces in the bottom row are from positive antiserum tested by Flag peptide ELISA. The traces labeled 293/Flag-GPR40 are data from a stable cell line. 293/Flag-EDG4 and 293/myc-EDG4 are data from transiently transfected cells.

FIG. 20 is data from FACS analysis of mouse immune sera where EDG4 CHO stable cell line was the immunogen. The dark black trace is from a CHO/GPR40 cell line, while the light gray trace is from a CHO/EDG4 cell line.

FIG. 21 displays a screening assay of anti-EDG4 monoclonal antibodies from CHO/GPR40 and CHO/EDG4 cell lines.

FIG. 22 is a competitive binding assay using EDG4 antibody to block the receptor activation by the G-protein 5G3.

### DETAILED DESCRIPTION OF THE INVENTION

### Definitions

The abbreviation “GPCR” refers to G-protein Coupled Receptor, and as used herein encompasses the protein, amino acid sequence, and nucleotide sequence encoding for a G-protein coupled receptor.

The terms “heterologous protein”, “recombinant protein”, and “exogenous protein” can be used interchangeably in referring to aspects of this invention. These phrases refer to a polypeptide which is produced by recombinant DNA techniques, wherein DNA encoding the polypeptide is inserted into a suitable expression vector which is in turn used to transform a host cell to produce the heterologous protein.

As used herein, “heterologous G protein coupled receptor” (e.g., a heterologous adenosine receptor) is a receptor encoded by heterologous DNA. Upon expression of the heterologous DNA in a recombinant cell, the heterologous receptor is expressed in the recombinant cell. The term heterologous G protein coupled receptor, or GPCR, as used herein encompasses wildtype proteins (and the nucleotide sequences which encode for them) as well as all variants or mutants, whether those variations or mutations are naturally-occurring or created through genetic or molecular engineering.

The term “signal transduction” encompasses the processing of physical or chemical signals from the extracellular environment through the cell membrane and into the cell, and may occur through one or more of several mechanisms, such as activation/inactivation of enzymes (such as proteases, or other enzymes which may alter phosphorylation patterns or other post-translational modifications), activation of ion channels or intracellular ion stores, effector enzyme activation via guanine nucleotide binding protein intermediates, formation of inositol phosphate, activation or inactivation of adenylyl cyclase, direct activation (or inhibition) of a transcriptional factor and/or activation.

The term “functionally” couples to (as in a receptor that is “functionally integrated into a signaling pathway in a cell” or “functionally integrated into an endogenous yeast signaling pathway” or “functionally expressed by a host cell”) refers to the ability of a receptor to bind to modulators and transduce that binding event into a signal using components of a signaling pathway of the cell. For example, GPCR which is functionally integrated into an endogenous pheromone response or signaling pathway of a yeast cell is expressed on the surface of the yeast cell, couples to a G protein within the yeast cell and transduces a signal in that yeast cell upon binding of a modulator to the receptor.

The term “modulation”, as in “modulation of a (heterologous) G protein coupled receptor” and “modulation of a signal transduction activity of a receptor protein” encompasses, in its various grammatical forms, induction and/or potentiation, as well as inhibition and/or downregulation of receptor activity and/or one or more signal transduction pathways downstream of a receptor.

An “oligonucleotide”, as used herein, refers to a stretch of nucleotide residues which preferably has a sufficient number of bases to be used as an oligomer, amplimer or probe in a polymerase chain reaction (PCR). Oligonucleotides are prepared synthetically or from genomic or cDNA sequences and are preferably used to amplify, reveal, or confirm the presence of a similar DNA or RNA in a particular cell or tissue. Oligonucleotides or oligomers comprise portions of a DNA sequence having at least about 10 nucleotides and as many as about 35 nucleotides, preferably about 25 nucleotides.

“Probes” refers to oligonucleotides derived from naturally occurring recombinant single- or double-stranded nucleic acids or may be chemically synthetic. Oligonucleotides are useful in detecting the presence of complementary identical or similar sequences. Probes may be labeled with reporter molecules using nick translation, Klenow fill-in reaction, PCR or other methods well known in the art. Nucleic acid probes may be used in Southern, Northern or in situ hybridization to determine whether DNA or RNA encoding a certain protein is present in a cell type, tissue, or organ.

A “fragment of a polynucleotide” is a nucleic acid that comprises all or any part of a given nucleotide molecule. An exemplary fragment is about 6 kb in length, preferably having fewer nucleotides than about 6 kb, more preferably having fewer than about 1 kb.

“Reporter molecules” include chemical, radionucleic, enzymatic, fluorescent, chemiluminescent, or chromogenic agents which associate with a particular nucleotide sequence, receptor, or amino acid sequence, thereby establishing the presence of or quantifying the expression of a certain sequence, receptor or agent binding to the receptor.

“Chimeric” oligonucleotides may be constructed by introducing all or part of a nucleotide sequence of this invention into a vector containing additional nucleic acid sequence which might be expected to change any one or several of the following GPCR characteristics: cellular location, distribution, ligand-binding affinities, interchain affinities, degradation/turnover rate, signaling, etc. Similarly, chimeric peptides are GPCR amino acid sequences which have been constructed to contain additional amino acid sequences which might be expected to change any one or several of the following GPCR characteristics: cellular location, distribution, ligand-binding affinities, interchain affinities, degradation/turnover rate, signaling, etc.

“Active”, with respect to a GPCR, refers to those forms, fragments, or domains of a GPCR polypeptide which retain the biological and/or antigenic activity of a GPCR polypeptide.

“Naturally occurring GPCR polypeptide” refers to a polypeptide produced by cells which are not genetically engineered and specifically contemplates various polypeptides arising from post-translational modifications of the polypeptide including but not limited to acetylation, carboxylation, glycosylation, phosphorylation, lipidation and acylation.

“Derivative” refers to polypeptides which are chemically modified by techniques such as ubiquitination, labeling, pegylation (derivatization with polyethylene glycol), insertion or substitution of amino acids such as ornithine which do not normally occur in human proteins, or one or more amino acids from the wild type sequence.

“Conservative amino acid substitutions” result from replacing one amino acid with another having similar structural and/or chemical properties, such as the replacement of a leucine with an isoleucine or valine, an aspartate with a glutamate, or a threonine with a serine.

A “signal sequence” or “leader sequence” can be used, when desired, to direct the polypeptide through a membrane of a cell. Such a sequence may be naturally present on polypeptides produced using the vectors and methods of the present invention or provided from heterologous sources by recombinant DNA techniques.

“Inhibitor” is any substance which retards or prevents chemical or physiological reactions or responses. Common inhibitors include but are not limited to antisense molecules, antibodies, and antagonists.

“Standard expression” is a quantitative or qualitative measurement for comparison. It is based on a statistically appropriate number of normal samples and is created to use as a basis of comparison when performing diagnostic assays, running clinical trials, or following patient treatment profiles.

“Animal”, as used herein, includes human, domestic (e.g., cats, dogs, etc.), agricultural (e.g., cows, horses, sheep, etc.) or test species (e.g., mouse, rat, rabbit, etc.).

“Stringent conditions” refers to conditions that allow for the hybridization of essentially complementary nucleic acid sequences. For instance, such conditions will generally allow hybridization of sequence with at least about 85% sequence identity, preferably with at least about 90% to 95% sequence identity, more preferably about 91% sequence identity, about 92% sequence identity, about 93% sequence identity, about 94% sequence identity, more preferably with at least about 95% to 99% sequence identity, preferably about 96% sequence identity, about 97% sequence identity, about 98% sequence identity, still more preferably about 99% sequence identity, or about 100% sequence identity to the complementary nucleic acid sequences.

“Recombinant cells” encompasses one or more individual cells as well as to a recombinant cell line in which the cells are expressing a heterologous protein.

The term “extracellular signal” encompasses molecules and changes in the cellular environment that are transduced intracellularly via cell surface proteins that interact, directly or indirectly, with the extracellular signal. An extracellular signal or effector molecule includes any compound or substance that in some manner alters the activity of a cell surface protein. Examples of such signals include, but are not limited to, molecules such as acetylcholine, growth factors and hormones, lipids, sugars and nucleotides that bind to cell surface receptors and modulate the activity of such receptors. The term, “extracellular signal” also includes as yet unidentified substances that modulate the activity of a cellular receptor, and thereby influence intracellular functions. Such extracellular signals are potential pharmacological agents that may be used to treat specific diseases by modulating the activity of specific cell surface receptors.

The term “functional assays” as used herein encompasses those assays that take advantage of certain aspects of GPCR protein activity or behavior under particular conditions, for example, the activation of a G protein upon binding of a ligand to the GPCR.

The term “selectively binds” as used herein refers to a compound (e.g., an antibody, a peptide, a lipid or a small organic molecule) that binds to a native polypeptide or to a chimeric polypeptide preferentially relative to other unrelated polypeptides. A compound selectively binds to the native polypeptide or a chimeric polypeptide of the invention if it has at least a 10%, preferably at least a 25%, at least a 50%, at least a 75%, at least a 90%, at least a 95%, or at least a 100% higher affinity and/or avidity for the native polypeptide or chimeric polypeptide than an unrelated polypeptide.

### Introduction

G protein coupled receptors (hereinafter termed “GPCRs”) comprise a large superfamily of receptors. GPCRs were originally defined as receptors that transduce signals from the extracellular compartment to the interior through biochemical processes involving GTP-binding proteins. Molecular cloning of the first receptor genes suggested protein structures with seven transmembrane α-helical domains (hence “7TM receptors”). Typical GPCRs do share a common structural motif of seven transmembrane helical domains, but some GPCRs are instead single-spanning transmembrane receptors for cytokines such as erythropoietin or insulin, or multi-polypeptide receptors such as the collagen receptor.

As used herein, “GPCR protein” and “a GPCR” refers to a protein in which one response to the binding of a ligand to the GPCR is the activation of a G protein. This term also encompasses the amino acid and nucleotide sequences of these proteins. “A GPCR” is meant to include both the singular and plural forms of the phrase, i.e., “a GPCR” may refer to one or more GPCR molecules.

Modern crystallography and mutational analyses show that GPCRs are versatile receptors for a wide range of extracellular messengers, including biogenic amines, purines and nucleic acid derivatives, lipids, peptides and proteins, odorants, pheromones, tastants, ions like calcium and protons, and photons (in the case of rhodopsin). GPCRs can form homo- and heterodimers, as well as complex receptosomes, which in some cases can incorporate additional intra- and extracellular soluble and transmembrane proteins.

GPCRs play a vital role in the signaling processes that control cellular metabolism, cell growth and motility, inflammation, neuronal signaling, and blood coagulation. G protein coupled receptor proteins also serve as targets for molecules such as hormones, neurotransmitters and physiologically active substances. Thus, GPCRs are a major target for drug action and development.

High Expression Vectors

In a first aspect, the invention provides a vector which facilitates high levels of expression of GPCR proteins in a cell line. Native GPCR proteins are expressed in levels numbering in the upper hundreds to low thousands of copies per cell. Since most molecular and cell biology techniques, such as screening assays and raising antibodies, cannot be conducted with such low levels of protein, high expression vectors are needed to produce proteins on the order of tens of thousands to millions of copies per cell. The vectors encompassed by the instant invention overcome the hurdle to GPCR research posed by the low expression of native GPCR proteins by facilitating high levels of expression of GPCR proteins in mammalian cells.

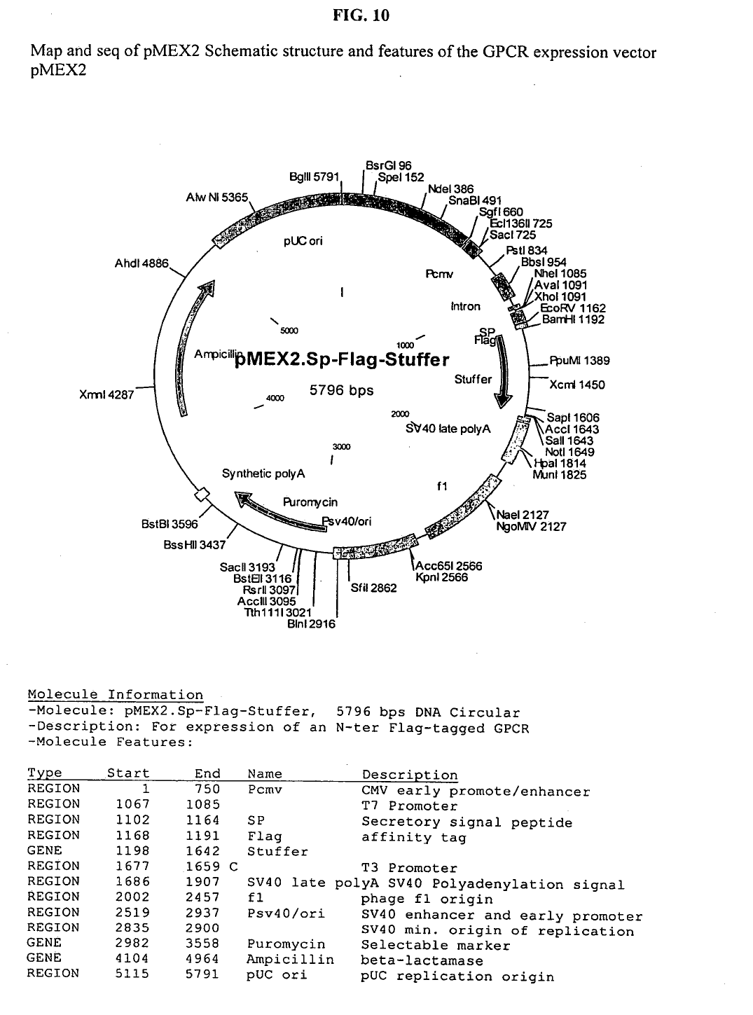

In an exemplary embodiment, according to FIG. 10, the invention provides a novel vector named pMEX2 for expression of GPCR proteins on the cytoplasmic membrane of mammalian cells. As shown in FIG. 10, this vector includes a pUC origin and a beta-lactamase gene for replication and ampicillin selection of the plasmid in bacteria. A puromycin resistance marker for maintaining the plasmid in mammalian cells is also included in certain embodiments of this vector. Expression of the gene of interest is under control of a strong CMV promoter for high-level transcription activity.

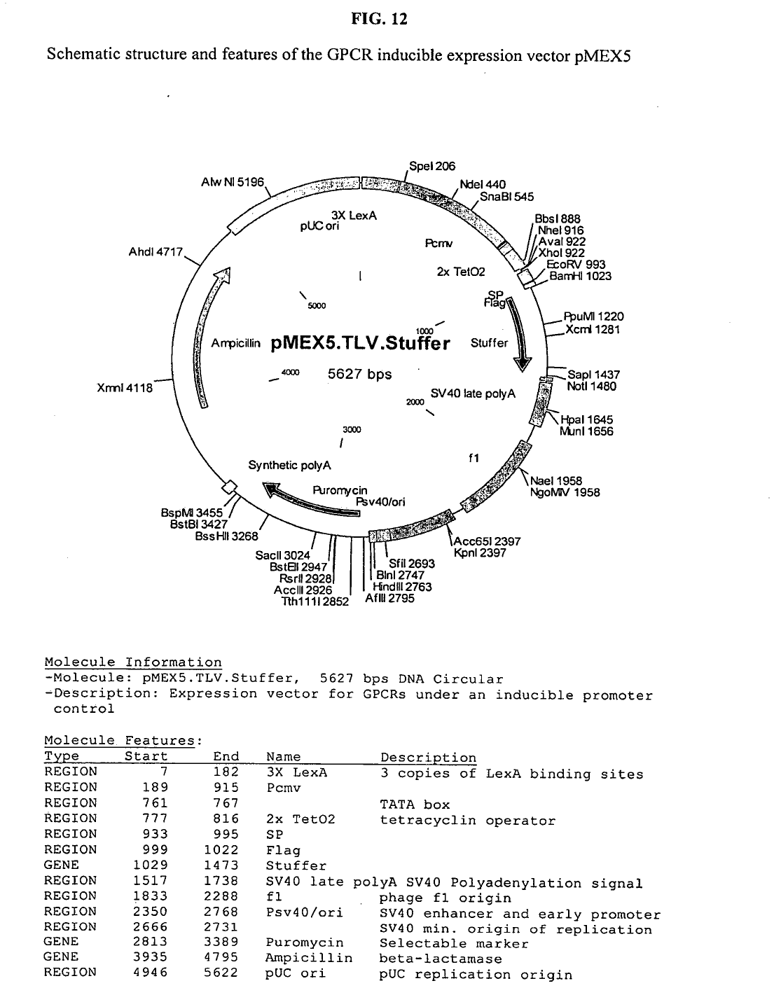

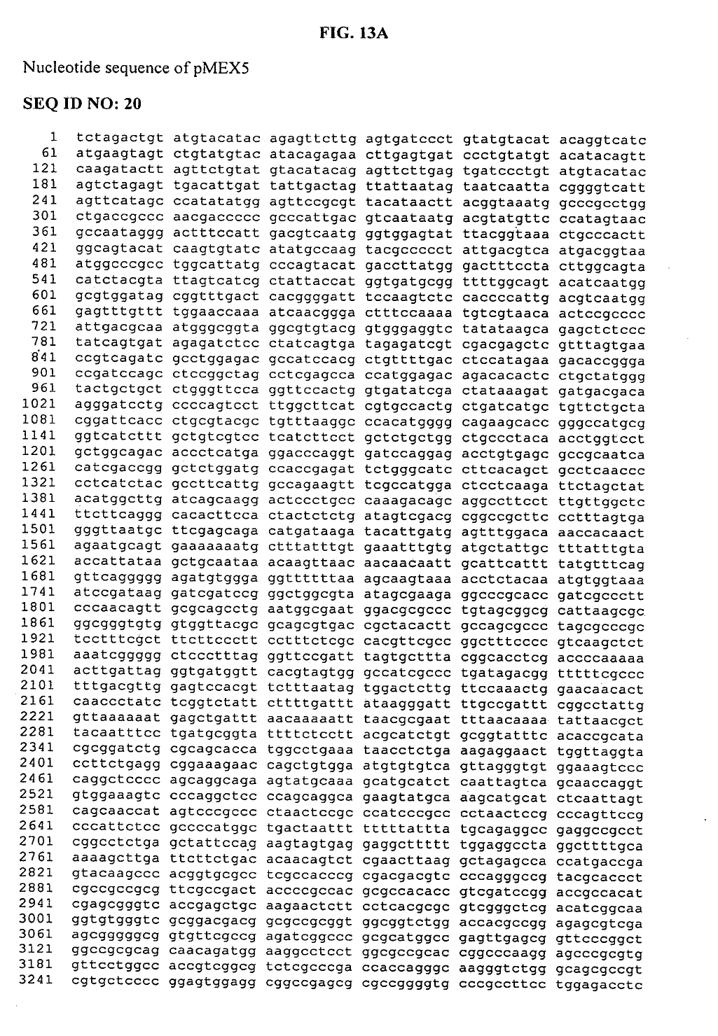

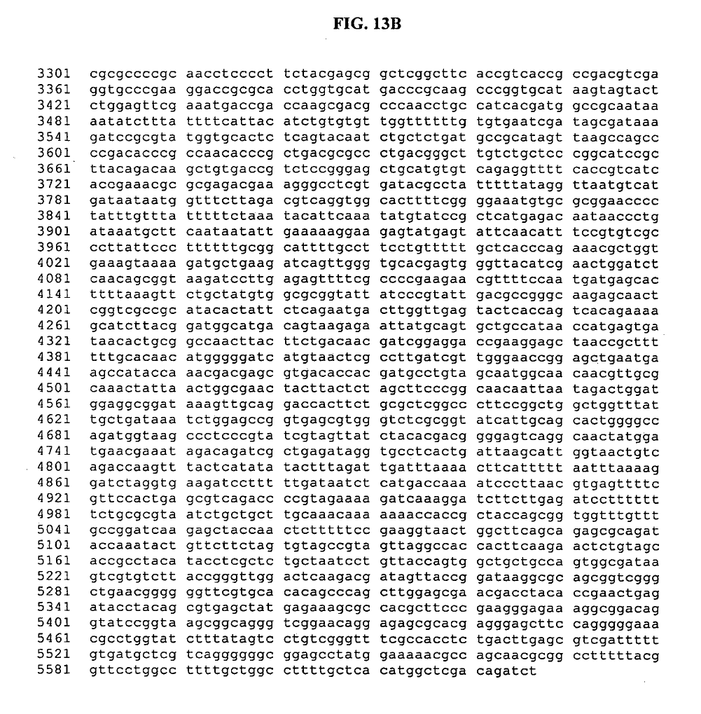

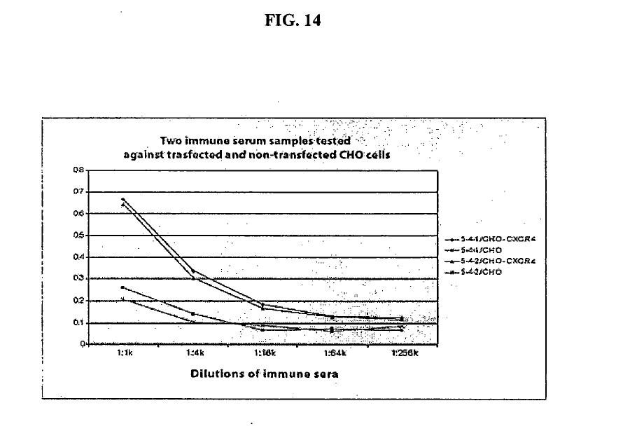

In another exemplary embodiment, according to FIG. 12, the invention provides a novel vector named pMEX5 for expression of GPCR proteins on the cytoplasmic membrane of mammalian cells. This vector allows for expression of GPCR proteins under control of an inducible promoter. As in pMEX2, pMEX5 includes a strong CMV promoter for high-level transcription activity, with the additional feature that the promoter is operably linked to a tetracycline operator, as shown in FIG. 12. In alternative embodiments of the invention, the inducible promoter may be selected from a chemical inducible promoter, such as a steroid-responsive promoter, a tissue responsive promoter, a promoter derived from the genome of mammalian cells, such as the metallothionein promoter, and a promoter derived from mammalian viruses, such as the retrovirus long terminal repeat, the adenovirus late promoter, and the vaccinia virus 7.5K promoter. Promoters produced by recombinant DNA or synthetic techniques may also be used to provide for transcription of a polypeptide-encoding nucleotide sequence.

In both the vectors pictured in FIG. 10 and FIG. 12 respectively, the vectors include Kozak consensus sequence for optimal translation initiation and an SV40 late polyadenylation signal to promote stability in the transcripts. Also included in the vectors are signal sequences (labeled “SP” in FIG. 10 and FIG. 12) which provide efficient delivery of the translated protein to the membrane of the cell. It is intended that the exemplary signal sequences shown in FIG. 10 and FIG. 12 are not meant to be limiting, and that the instant invention encompasses all possibilities of signal sequences which are effective in targeting the translated protein to the cell membrane.

In an exemplary embodiment, the invention provides binding sites for the bacteriophage DNA binding protein LexA that are engineered just upstream of the promoter sequence of the expression vector. An expression vector engineered in this way will express a chimeric protein that includes the LexA DNA binding domain linked to an activation domain, such as that for the herpes simplex virus protein VP16. In principle, this combination of DNA binding site, DNA binding protein and activation domain can be manipulated by using DNA binding sites, DNA binding proteins and/or activation domains to strengthen the ability of the promoter to initiate and sustain transcription of downstream elements in the vector.

In one embodiment of the invention, the expression vector includes a nucleotide sequence for a GPCR protein selected from one of several possible GPCR families of proteins, including: anaphylatoxin, apelin, bombesin, cannabinoid, chemokine, free fatty acid, galanin, glucagon, glycoprotein hormone, leukotriene/lipoxin, lysophospholipid, melanin-concentrating hormone, melatonin, N-formylpeptide, neuromedin U, neuropeptide S, neuropeptide W/neuropeptide B, neuropeptide Y, opioid, platelet activating factor, prolactin releasing peptide, prostanoid, PTH, purinergic, tachykinin, trace amine, and urotensin.

In another embodiment of the invention, the expression vector includes a nucleotide sequence encoding for an “orphan” GPCR, which is a GPCR protein for which there is as of yet no known ligand. There are currently over two hundred GPCR proteins identified as orphan GPCRs. These orphan proteins may be implicated in a number of disease, such as cancer and inflammation associated with arthritis, and thus orphan GPCRs are of particular interest to the pharmaceutical and biotechnology industries.

In a further embodiment of the invention, the expression vector includes a nucleotide sequence encoding for a GPCR that is a member selected from: C3aR, APJ, BB1, BB3, GPR55, CCR1, CCR5, CCR7, CCR9, CMKLR1, CXCR3, CXCR4, FFA1, FFA2, GAL1, GAL2, GAL3, GHRH, TSH, ALX, BLT1, BLT2, CysLT1, LPA2, LPA3, MCH1, MT2, FPR1, NMU1, NPS, NPS(1), NPS(2), NPS Ile107, NPBW1, NPBW2, delta, kappa, mu, NOP, GPR37L1, GPR84, MRGX1, MRGX2, PSGR, PAF, PRP, DP, EP1, GPR44, PTH2, P2Y12, NK2, NK3, TA1, C5aR, PAR2.

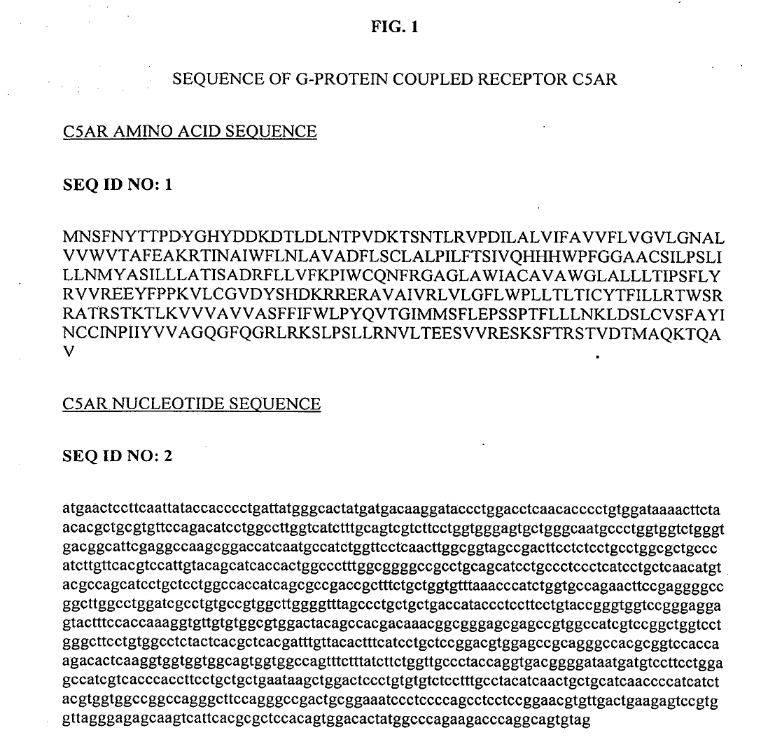

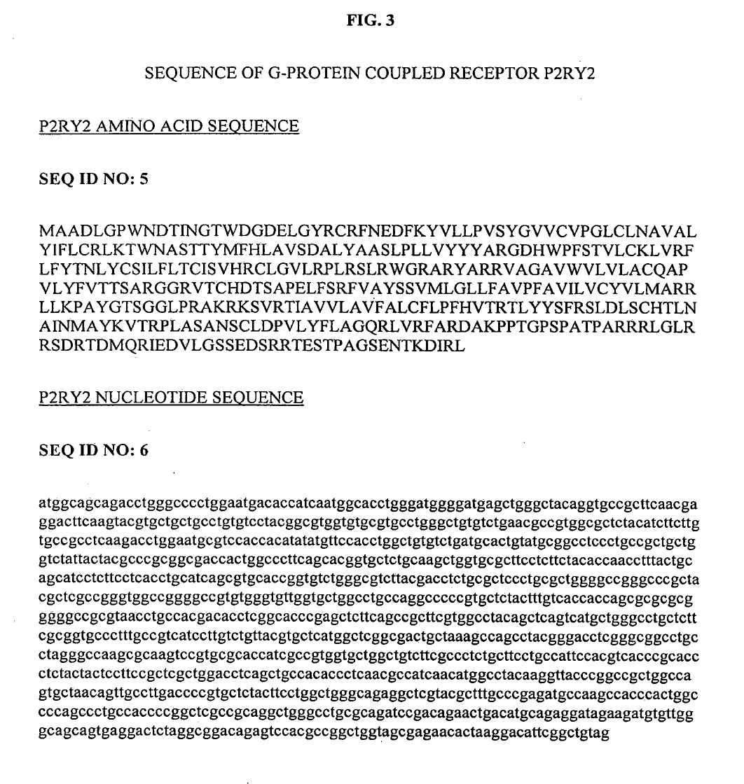

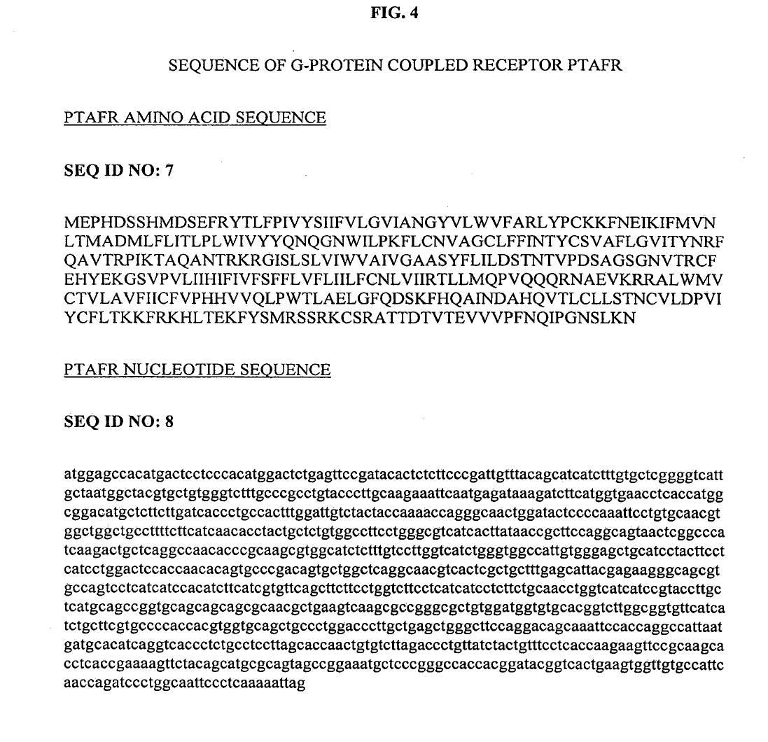

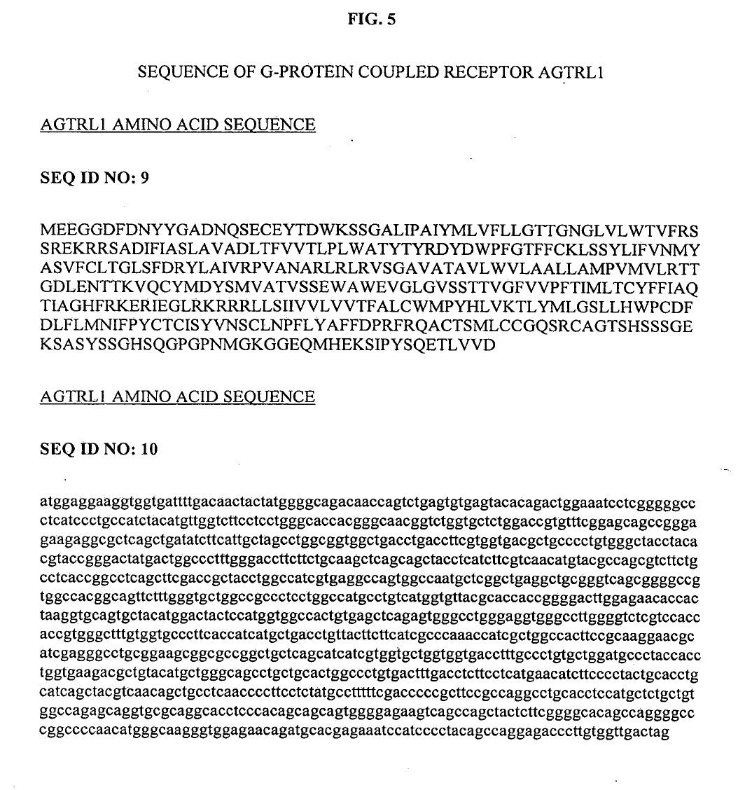

In a still further embodiment, the expression vector includes a GPCR protein which has an amino acid sequence that is a member selected from: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, and SEQ ID NO: 17, as pictured in FIG. 1 through FIG. 9.

In a further embodiment of the invention, the expression vector comprises a member selected from: SEQ ID NO: 19 and SEQ ID NO: 20. Vector maps graphically illustrating the expression vectors corresponding to these sequences are shown in FIG. 10 and FIG. 12 respectively. The expression vectors in one embodiment of the invention contain multiple restriction sites which can be used to insert a nucleotide sequence encoding for a GPCR.

In a further embodiment, the invention provides expression vectors that include nucleotide sequences for molecules to aid in the detection of GPCR. In a still further embodiment, such detection includes the ability to determine if the protein is expressed in the correct orientation on the cytoplasmic membrane of mammalian cells.

In a preferred embodiment, the invention provides expression vectors containing a pUC origin and a beta-lactamase gene for replication and ampicillin selection of the plasmid in bacteria. In a further embodiment of the invention, the expression vector can also include a puromycin resistance marker for the gene of interest, which is under control of a strong CMV promoter for high-level transcription activity.

In a preferred embodiment of the invention, a method is provided for creating expression vectors that enable expression of GPCRs, properly folded with appropriate post-translational modifications, with levels of expression of at least one million copies per cell on the cytoplasmic membrane. This level of expression is suitable for whole-cell immunization for raising antibodies as well as for functional and structural studies of the receptor.

### Kozak Sequence

Most eukaryotic mRNAs contain a short recognition sequence that facilitates the initial binding of mRNA to the small subunit of the ribosome. The consensus sequence for initiation of translation in vertebrates (also called Kozak sequence) is: ACCATG (see, e.g., SEQ ID NO: 19, position 1099-1104). More generally it is: GCCRCCATGG where R is a purine (A or G) (see, e.g., SEQ ID NO: 19, position 1096-1105). To improve expression levels, it may be advantageous to design the cloned insert according to Kozak's rules in the present invention.

### Recombinant Cells

In one aspect, the invention provides recombinant cell lines expressing GPCR proteins.

In a preferred embodiment of the invention, expression of GPCR proteins is governed by a high expression vector as described above. As used herein, the terms “recombinant cell line”, “cell line”, “recombinant cells” can be used interchangeably to refer to cells heterologously expressing an indicated protein.

In one embodiment of the invention, the recombinant cells express levels of GPCR of at least 150,000 protein molecules per cell. In a further embodiment of the invention, GPCR is expressed at a range of 200,000 copies to 2,000,000 copies per cell. In a still further embodiment of the invention, the GPCR protein is expressed at a range of 400,000 copies to 2,000,000 copies per cell. In a still further embodiment of the invention, the recombinant cells express GPCR protein at a range of 600,000 copies to 2,000,000 copies per cell. In a yet further embodiment of the invention, the recombinant cells express GPCR protein at a range of 800,000 copies to 2,000,000 copies per cell. In a preferred embodiment of the invention, the recombinant cells express GPCR protein at a range of 1,000,000 copies to 2,000,000 copies per cell. In another preferred embodiment, the cells express GPCR protein at a range of 1,500,000 copies to 2,000,000 copies per cell.

In yet another embodiment of the invention, the GPCR protein is derived from an animal. In a further embodiment of the invention, the GPCR protein is derived from a mammal, including rat, mouse or human.

In still another embodiment of the invention, the recombinant cell expressing the GPCR protein is derived from a cell line, which may as an example be selected from a Chinese hamster ovary (CHO) cell line, a human embryonic kidney cell line (HEK293T), a C6 glioma cell line, the RH7777 cell line, the SW480 cell line from human adenocarcinoma of the colon, the VS35 cell line, the 1321N1 cell line, and other cell lines that are known in the art to be amenable to stable or transient transfection with heterologous nucleic acids.

In one aspect, the invention provides a method of producing a cell line, which includes creating at least one expression vector selected from nucleotide sequence SEQ ID NO: 19 or SEQ ID NO: 20 and transfecting a host cell with the expression vector. The transfection of the host cell can either be such that it creates a stably transfected cell line or a transiently transfected cell line by methods known in the art.

In one embodiment of the invention, the stably or transiently transfected cell line is a mammalian cell line. In a preferred embodiment, the cell line is derived from a cell line selected from a group consisting of: CHO, HEK293T, C6, RH7777, SW480, VS35, and 1321N1. Mammalian cell lines are particularly preferred, because such cell lines ensure that the protein will receive the proper post-translational modifications before being transported to the cell membrane.

### GPCR-Expressing Recombinant Cells

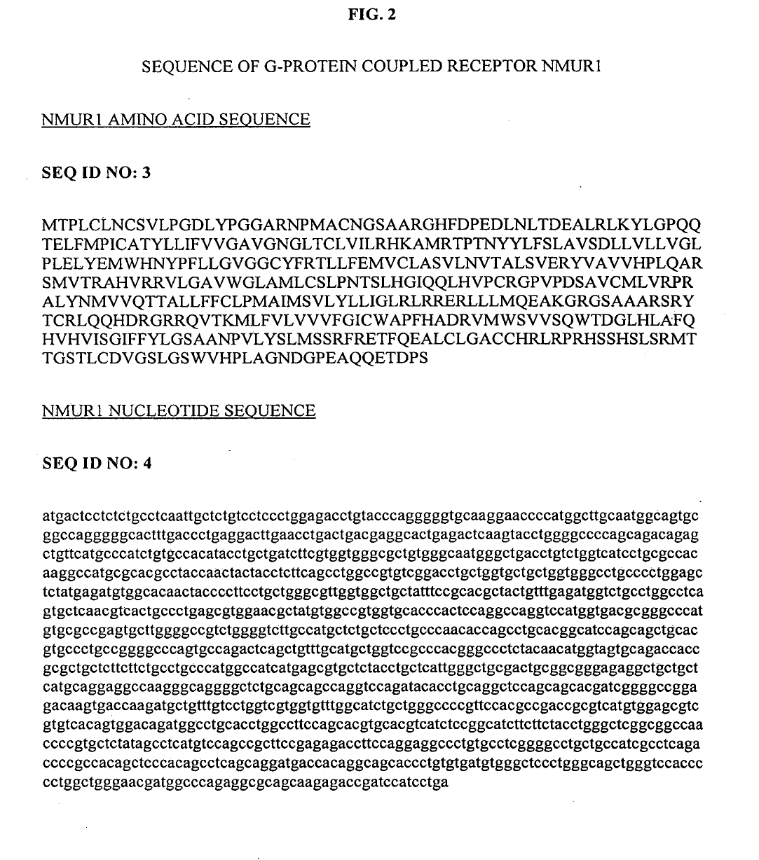

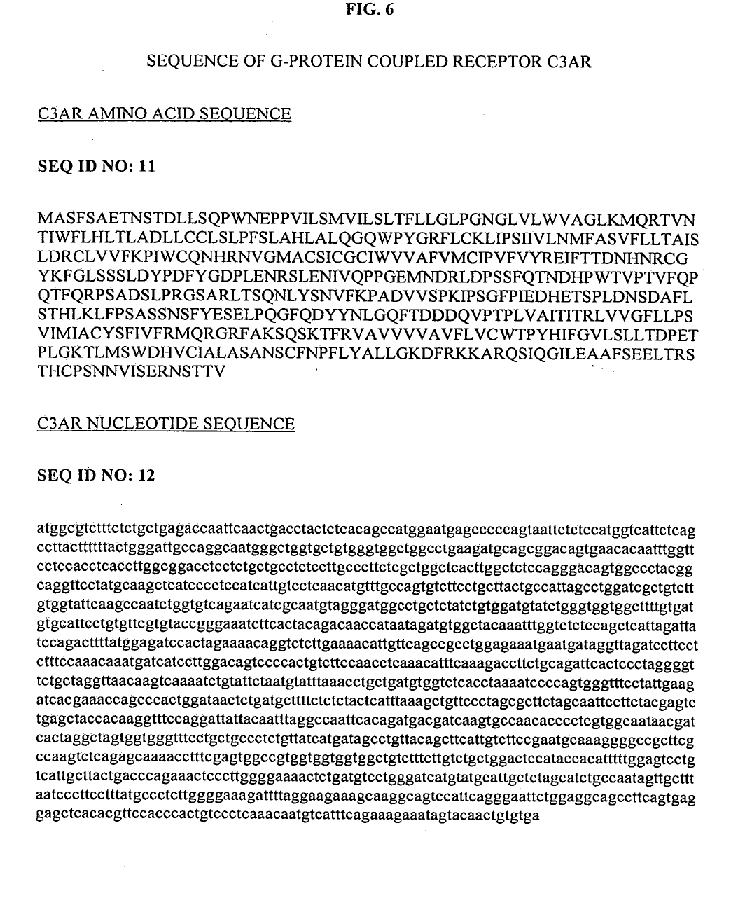

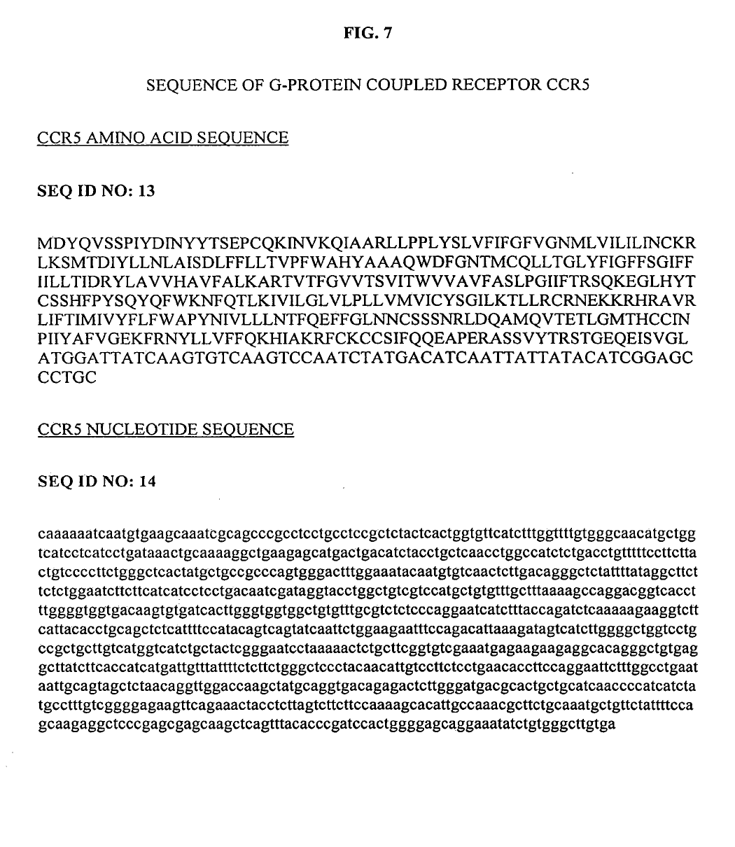

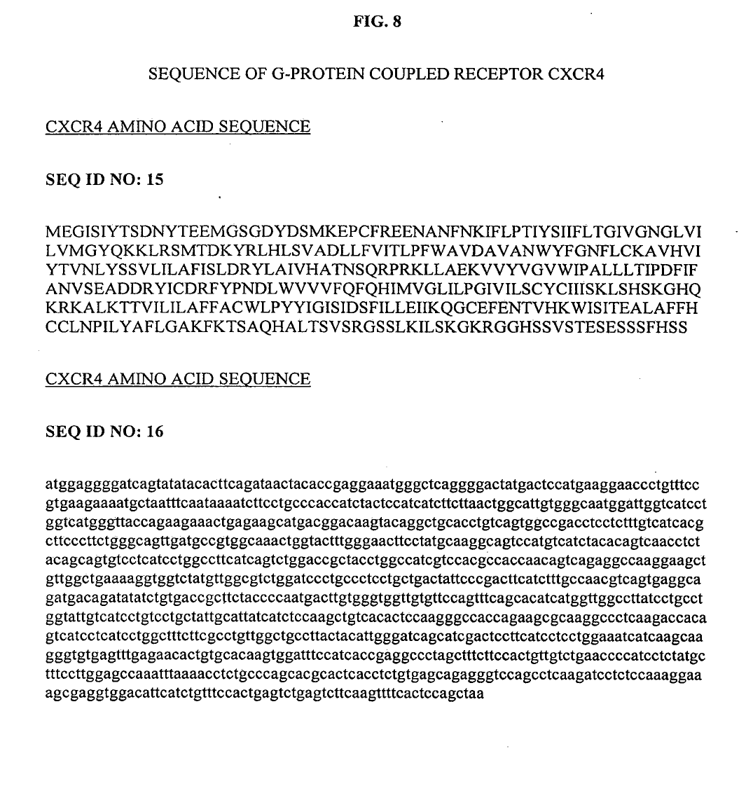

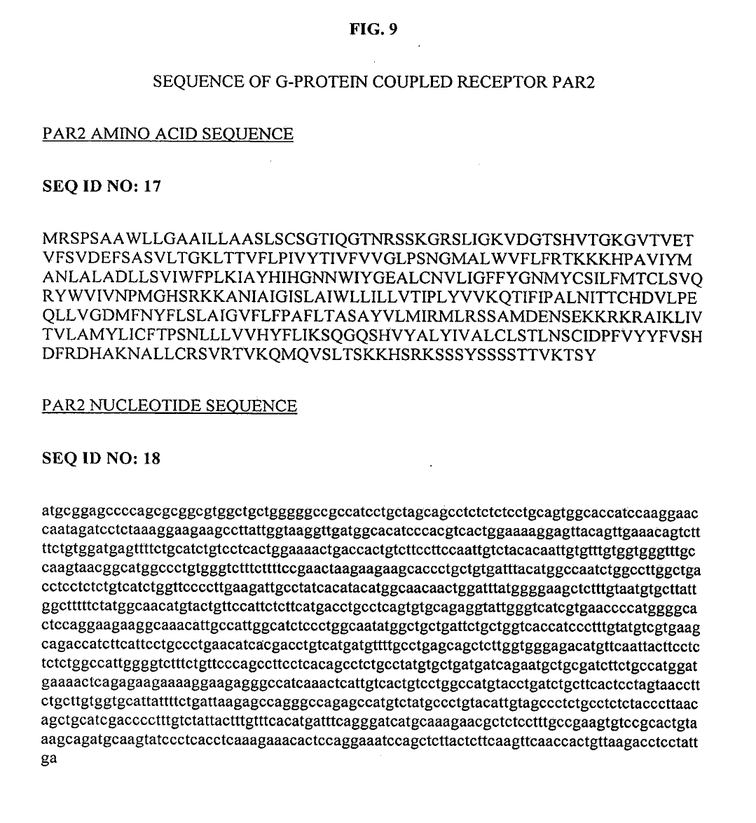

In one embodiment, the invention provides recombinant cells expressing a GPCR protein that has an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9. In a further embodiment of the invention, the GPCR protein is a member selected from C3aR (in accordance with FIG. 6), APJ (in accordance with Accession number NM_{—}005161), BB1 (in accordance with Accession number NM_{—}012799), BB3 (in accordance with Accession number NM_{—}001727), GPR55 (in accordance with Accession number NM_{—}005683), CCR1 (in accordance with Accession number NM_{—}000579), CCR5 (in accordance with FIG. 7), CCR7 (in accordance with Accession number NM_{—}001838), CCR9 (in accordance with Accession number NM_{—}006641), CMKLR1 (in accordance with Accession number NM_{—}004072), CXCR3 (in accordance with Accession number NM_{—}001504), CXCR4 (in accordance with FIG. 8), FFA1 (in accordance with Accession number NM_{—}005303), FFA2 (in accordance with Accession number NM_{—}005304), GAL1 (in accordance with Accession number NM_{—}001480), GAL2 (in accordance with Accession number NM_{—}003857), GAL3 (in accordance with Accession number NM_{—}003614), GHRH (in accordance with Accession number NM_{—}000823), TSH (in accordance with Accession number NM_{—}012888), ALX (in accordance with Accession number NM_{—}003857), BLT1 (in accordance with Accession number BC_{—}004545), BLT2 (in accordance with Accession number NM_{—}0193839.1), CysLT1 (in accordance with Accession number NM_{—}006639), LPA2 (in accordance with Accession number NM_{—}004724.4), LPA3 (in accordance with Accession number NM_{—}012152.1), MCH1 (in accordance with Accession number NM_{—}005297), MT2 (in accordance with Accession number NM_{—}005959), FPR1 (in accordance with Accession number NM_{—}002029), NMU1 (in accordance with FIG. 2), NPS (in accordance with Accession number NM_{—}175678), NPS(1) (in accordance with Accession number NM_{—}207172), NPS(2) (in accordance with Accession number NM_{—}207173), NPS Ile107 (in accordance with Accession number SNP591694), NPBW1 (in accordance with Accession number NM_{—}001014784 and NM_{—}005285), NPBW2 (in accordance with Accession number NM_{—}005286), delta (in accordance with Accession number NM_{—}012617), kappa (in accordance with Accession number L22001), mu (in accordance with Accession number L13069), NOP (in accordance with Accession number BC038433), GPR37L1 (in accordance with Accession number NM_{—}004767), GPR84 (in accordance with Accession number NM_{—}020370), MRGX1 (in accordance with Accession number NM_{—}147199), MRGX2 (in accordance with Accession number NM_{—}054030), PSGR (in accordance with Accession number NM_{—}030774), PAF (in accordance with Accession number NM_{—}000952), PRP (in accordance with Accession number NM_{—}004248), DP (in accordance with Accession number NM_{—}000953), EP1 (in accordance with Accession number NM_{—}000955), GPR44 (in accordance with Accession number NM_{—}004778), PTH2 (in accordance with Accession number NM_{—}005048), P2V12 (in accordance with Accession number NM_{—}022788), NK2 (in accordance with Accession number NM_{—}001057), NK3 (in accordance with Accession number NM_{—}175057), TA1 (in accordance with Accession number NM_{—}138327), C5aR (in accordance with FIG. 1), PAR2 (in accordance with FIG. 9).

In another embodiment of the invention, recombinant cells express a GPCR protein encoded by a nucleotide sequence selected from SEQ ID NO: 2, SEQ ID NO: 4, SEQ ID NO: 6, SEQ ID NO: 8, SEQ ID NO: 12, SEQ ID NO: 14, SEQ ID NO: 16, and SEQ ID NO: 18, as pictured in FIG. 1 through FIG. 9. In a further embodiment of the invention, transcription of the nucleotide sequence encoding a GPCR protein is operably linked to a promoter, such as the cytomegalovirus (CMV) promoter. In a still further embodiment of the invention, the promoter is itself operably linked to an inducible operator, for example a tetracycline operator.

In another embodiment of the invention, the recombinant cells express GPCR protein at a level of at least 150,000 copies per cell. In a further embodiment, the invention provides GPCR expression levels at a range of about 150,000 copies and 2,000,000 copies per cell. In a still further embodiment, the invention provides GPCR expression levels at a range of about 200,000 copies and 2,000,000 copies per cell. In a still further embodiment, the invention provides GPCR expression levels at a range of about 300,000 copies and 2,000,000 copies per cell. In a further embodiment, the invention provides GPCR expression levels at a range of about 400,000 copies and 2,000,000 copies per cell. In a still further embodiment, the invention provides GPCR expression levels at a range of about 500,000 copies and 2,000,000 copies per cell. In a still further embodiment, the invention provides GPCR expression levels at a range of about 600,000 copies and 2,000,000 copies per cell. In a still further embodiment, the invention provides GPCR expression levels at a range of about 700,000 copies and 2,000,000 copies per cell. In a still further embodiment, the invention provides GPCR expression levels at a range of about 800,000 copies and 2,000,000 copies per cell. In a still further embodiment, the invention provides GPCR expression levels at a range of about 900,000 copies and 2,000,000 copies per cell. In a still further embodiment, the invention provides GPCR expression levels at a range of about 1,000,000 copies and 2,000,000 copies per cell. In a still further embodiment, the invention provides GPCR expression levels at a range of about 1,500,000 copies and 2,000,000 copies per cell. In a still further embodiment, the invention provides GPCR expression levels at a range of about 1,750,000 copies and 2,000,000 copies per cell.

In one aspect, the invention provides a recombinant cell line stably expressing a GPCR protein that has an amino acid sequence selected from SEQ ID NO: 1, SEQ ID NO:3, SEQ ID NO: 5, SEQ ID NO: 7, SEQ ID NO: 9, SEQ ID NO: 11, SEQ ID NO: 13, SEQ ID NO: 15, and SEQ ID NO: 17. It will be understood by those of skill in the art that these amino acid sequences encompass the nucleic acid sequences which encode for them.

### Nucleotide and Amino Acid Sequences Encoding for GPCR

In preferred embodiments, the instant invention uses nucleotide and amino acid sequences encoding for GPCR proteins in functional and cell-based assays and to produce recombinant cell lines. The nucleotide sequences encoding GPCRs (or their complements) have numerous applications in techniques known to those skilled in the art of molecular biology. These techniques include their use: as hybridization probes, in the construction of oligomers for PCR, for chromosome and gene mapping, in the recombinant production of GPCR, and in generation of antisense DNA or RNA, their chemical analogs and the like. Uses of nucleotides encoding a GPCR disclosed herein are exemplary of known techniques and are not intended to limit their use in any technique known to a person of ordinary skill in the art. Furthermore, the nucleotide sequences disclosed herein may be used in molecular biology techniques that have not yet been developed, provided the new techniques rely on properties of nucleotide sequences that are currently known, e.g., the triplet genetic code, specific base pair interactions, etc.

It will be appreciated by those skilled in the art that as a result of the degeneracy of the genetic code, a multitude of GPCR-encoding nucleotide sequences may be produced. Some of these will bear only minimal homology to the nucleotide sequence of the known and naturally occurring GPCR. The invention has specifically contemplated each and every possible variation of nucleotide sequence that could be made by selecting combinations based on possible codon choices. These combinations are made in accordance with the standard triplet genetic code as applied to the nucleotide sequence of naturally occurring GPCR, and all such variations are to be considered as being specifically disclosed.

Although the nucleotide sequences which encode a GPCR, its derivatives or its variants are preferably capable of hybridizing to the nucleotide sequence of the naturally occurring GPCR polynucleotide under stringent conditions, it may be advantageous to produce nucleotide sequences encoding GPCR polypeptides or their derivatives which posses a substantially different codon usage. Codons can be selected to increase the rate at which expression of the peptide occurs in a particular prokaryotic or eukaryotic expression host in accordance with the frequency with which particular codons are utilized by the host. Other reasons for substantially altering the nucleotide sequence without altering the encoded amino acid sequence include the production of RNA transcripts with certain desirable properties, such as an increased half-life or greater specificity than is possible with the naturally occurring sequence.

Nucleotide sequences encoding GPCR polypeptides may be joined to a variety of other nucleotide sequences by means of well established recombinant DNA techniques. Useful nucleotide sequences for joining to GPCR-encoding polynucleotides include an assortment of cloning vectors such as plasmids, cosmids, lambda phage derivatives, phagemids, and the like. Vectors of interest include expression vectors, replication vectors, probe generation vectors, sequencing vectors, etc. In general, vectors of interest may contain an origin of replication functional in at least one organism, convenient restriction endonuclease sensitive sites, and selectable markers for one or more host cell systems.

It will be recognized that many deletional or mutational analogs of GPCR polynucleotides will be effective hybridization probes for GPCR polynucleotides. Accordingly, the invention relates to nucleic acid sequences that hybridize with such GPCR encoding nucleic acid sequences under stringent conditions.

Hybridization conditions and probes can be adjusted in well-characterized ways to achieve selective hybridization of human-derived probes. Exemplary stringent conditions, include a buffer containing 1 mM EDTA, 0.5 M NaHPO₄ (pH 7.2), 7% (w/v) SDS.

Nucleic acid molecules that will hybridize to GPCR polynucleotides under stringent conditions can be identified functionally. Without limitation, examples of hybridization probes include probes and primers used for identifying tissues that express GPCR, measuring mRNA levels, for instance to identity a sample's tissue type or to identify cells that express abnormal levels of GPCR, and detecting polymorphisms of GPCR.

It is possible to produce a DNA sequence, or portions thereof, entirely by synthetic chemistry. After synthesis, the nucleic acid sequence can be inserted into any of the many available DNA vectors and their respective host cells using techniques known in the art. Moreover, synthetic chemistry may be used to introduce mutations into a nucleotide sequence. A portion of sequence in which a mutation is desired can also be synthesized and recombined with longer portion of an existing genomic or recombinant sequence.

GPCR polynucleotides may be used to produce a purified oligo- or polypeptide using well known methods of recombinant DNA technology. The oligopeptide may be expressed in a variety of host cells, either prokaryotic or eukaryotic. Host cells may be from the same species from which the nucleotide sequence was derived or from a different species. Advantages of producing an oligonucleotide by recombinant DNA technology include obtaining adequate amounts of the protein for purification and the availability of simplified purification procedures.

Sequences encoding GPCR can be synthesized, in whole or in part, using chemical methods well known in the art. Alternatively, GPCR itself can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer). Optionally, fragments of GPCR can be separately synthesized and combined using chemical methods to produce a full-length molecule.

The newly synthesized peptide can be substantially purified by preparative high performance liquid chromatography. The composition of a synthetic GPCR can be confirmed by amino acid analysis or sequencing. Additionally, any portion of the amino acid sequence of GPCR can be altered during direct synthesis and/or combined using chemical methods with sequences from other proteins to produce a variant polypeptide or a fusion protein.

As will be understood by those of skill in the art, it may be advantageous to produce GPCR polynucleotides possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences referred to herein can be engineered using methods generally known in the art to alter GPCR polynucleotides for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Similarly, the polypeptide sequences referred to herein can be engineered or modified, preferably with post-translational modification that occur in the naturally occurring polypeptides, such as glycosylation.

### Antibodies

In one aspect, the invention provides an antibody or antigen binding fragment that specifically binds to a structural feature of a GPCR protein. Such an antibody or antigen binding fragment is raised against an immunogen, which in a further aspect of the invention is a cell line expressing between 150,000 and 2,000,000 copies of GPCR protein per cell. In one embodiment of the invention, the antibody or antigen binding fragment is a monoclonal antibody or antigen binding fragment.

In one embodiment of the invention, the antibody or antigen binding fragment binds to a structural feature of a GPCR protein which is a member of a GPCR family selected from anaphylatoxin, apelin, bombesin, cannabinoid, chemokine, free fatty acid, galanin, glucagon, glycoprotein hormone, leukotriene/lipoxin, lysophospholipid, melanin-concentrating hormone, melatonin, N-formylpeptide, neuromedin U, neuropeptide S, neuropeptide W/neuropeptide B, neuropeptide Y, opioid, platelet activating factor, prolactin releasing peptide, prostanoid, PTH, purinergic, tachykinin, trace amine, and urotensin.

In a further embodiment, the invention provides an antibody or antigen binding fragment which is raised against a cell line expressing a member of a GPCR family selected from C3aR, APJ, BB1, BB3, GPR55, CCR1, CCR5, CCR7, CCR9, CMKLR1, CXCR3, CXCR4, FFA1, FFA2, GAL1, GAL2, GAL3, GHRH, TSH, ALX, BLT1, BLT2, CysLT1, LPA2, LPA3, MCH1, MT2, FPR1, NMU1, NPS, NPS(1), NPS(2), NPS Ile107, NPBW1, NPBW2, delta, kappa, mu, NOP, GPR37L1, GPR84, MRGX1, MRGX2, PSGR, PAF, PRP, DP, EP1, GPR44, PTH2, P2Y12, NK2, NK3, TA1, C5aR, and PAR2. In a still further embodiment, the antibody or antigen binding fragment recognizes an epitope on a GPCR protein selected from C3aR, APJ, BB1, BB3, GPR55, CCR1, CCR5, CCR7, CCR9, CMKLR1, CXCR3, CXCR4, FFA1, FFA2, GAL1, GAL2, GAL3, GHRH, TSH, ALX, BLT1, BLT2, CysLT1, LPA2, LPA3, MCH1, MT2, FPR1, NMU1, NPS, NPS(1), NPS(2), NPS Ile107, NPBW1, NPBW2, delta, kappa, mu, NOP, GPR37L1, GPR84, MRGX1, MRGX2, PSGR, PAF, PRP, DP, EP1, GPR44, PTH2, P2Y12, NK2, NK3, TA1, C5aR, and PAR2.

In another embodiment, the invention provides an antibody or antigen binding fragment which is raised against a cell line expressing a member of GPCR family having an amino acid sequence selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, and SEQ ID NO: 17.

In a further embodiment, the invention provides an antibody or antigen binding fragment that binds to a structural feature of a GPCR protein and is raised against an immunogen which is a cell line expressing at least about 150,000 copies of a GPCR protein per cell. In a preferred embodiment, the cell line expresses about 500,000 copies of a GPCR protein per cell. In a particularly preferred embodiment, the cell line expresses between about 1,000,000 and about 2,000,000 copies of a GPCR protein per cell.

In one aspect, the invention provides a method for producing monoclonal antibodies for a GPCR protein. This method includes immunizing a test animal with at least one cell line expressing a member of a GPCR family. In accordance with the invention, this cell line expresses at least about 150,000 copies of said GPCR protein per cell. The test animal is induced to produce hybridomas, which are isolated. The method also includes screening for monoclonal antibodies using one or more cell-based assay systems. In one embodiment of the invention, the cell-based assay systems used to screen for monoclonal antibodies are selected from a group consisting of: FACS, ELISA, calcium imaging, FLIPR, multiplex ligand binding, and electrophysiology.

In one embodiment of the invention, monoclonal antibodies are induced in a test animal selected from a group comprising: rabbit, mouse, rat, pig, dog, monkey and goat. In a preferred embodiment, the invention provides a method of immunizing the test animal with whole cells expressing said member of GPCR family. In accordance with the invention, the whole cells used to immunize the test animal express at least 150,000 copies of the member of the GPCR family per cell.

GPCRs have traditionally been good drug targets for small molecule compounds and peptides. The field of antibody therapeutics for GPCRs is still in early-stage clinical trials. However, cases where small ligands can not be obtained such as for example, Family ii GPCRs, monoclonal antibodies can provide a viable alternative approach, Monoclonal antibodies may bind and lock GPCR in its active form and function as agonists. In addition, since the extracellular domains of GPCRs are more diverse than the rest of GPCR proteins including the transmembrane domains that small molecule compounds typically bind, monoclonal antibodies may bind to GPCRs more specifically than small molecules and thus can better distinguish subtle sequence and structural differences within sub-family members. Since GPCRs are also known to be overexpressed in many tumors, an advantage of GPCR antibody therapeutics is their ability to act as targeting moieties, guiding specific and accurate destruction of cancer cells.

### Kits

In one aspect, the invention provides a kit for high throughput purification and quantification of a plurality of recombinant proteins of one or more members of GPCR family. The kit includes a vector for expressing said recombinant proteins in host cells, wherein said vector comprises SEQ ID NO: 19 or 20, an affinity chromatography resin, a proteolytic enzyme, an internal quantification standard, a matrix for MALDI-TOF mass spectrometry, and instructions for use. In one embodiment, the invention further provides a kit that also includes at least one buffer selected from the group consisting of a lysis buffer; a denaturing buffer; an affinity chromatography binding buffer; an affinity chromatography washing buffer; an affinity chromatography elution buffer; and a proteolytic digestion buffer.

In another embodiment, the invention provides a kit for high throughput purification and quantification that includes at least one multi-well plate. In yet another embodiment, the invention provides a kit for high throughput purification and quantification which includes a partially or fully automated high throughput purification and quantification system.

In a further embodiment, the invention provides a kit which includes a vector that induces expression of one or more members of one or more GPCR families at a level of at least about 150,000 copies per cell. In a preferred embodiment, the vector induces between 150,000 and 2,000,000 copies of the GPCR protein per cell.

### Screening Methods

In one aspect, the invention provides methods for screening for therapeutic candidates. These methods include the use of a recombinant cell expressing a GPCR protein, where a test entity is contacted with the recombinant cell, and binding is detected between the test entity and the GPCR protein. As an embodiment of the invention, specific binding activity of the test entity to the GPCR protein identifies that test entity as a therapeutic candidate. The recombinant cell in this method expresses at least about 150,000 copies of the GPCR protein per cell. In a further embodiment of the invention, the test entity is contacted with a membrane extract of said recombinant cell. In a preferred embodiment of the invention, the method of screening for a therapeutic candidate employs a high throughput screen for detecting binding of the test entity to the GPCR protein. In a particularly preferred embodiment, the high throughput screen is partially or fully automated.

In one embodiment, the method for screening for therapeutic candidates includes a detection of binding of the test entity to the GPCR protein by means of a fluorescent, chemical, radiological, or enzymatic reporter molecule.

In an embodiment of the invention, the therapeutic candidate is screened for the treatment of cancer or of an illness associated with inflammation. In a preferred embodiment of the invention, the therapeutic candidate is screened for treatment of breast cancer.

In one aspect, the invention provides a method for identifying DNA sequences encoding a member of a GPCR family, which includes probing a cDNA library or a genomic library with a labeled probe with a nucleotide sequence selected from SEQ ID NO: 2, 4, 6, 8, 10, 12, 14, 16, and 18. DNA sequences from the library that are able to hybridize to the probe under stringent conditions are thus identified as encoding a member of a GPCR family. In an embodiment of the invention, the cDNA library or genomic library is derived from human tissue. In a particularly preferred embodiment, the human tissue used to create the cDNA or genomic library includes cancerous cells.

In one embodiment, the invention provides methods of using recombinant cell lines expressing the vectors of the present invention to prepare cDNA libraries of GPCRs. Such high expressing cells will be rich in GPCRs, which can in one embodiment of the invention be screened by low-stringency hybridization, or, alternatively, used in a polymerase chain reaction for amplification of candidate genes using degenerate polymers. Proof-of-function can obtained after the expression of the cloned receptor in heterologous cells with an elicited agonist response.

The compounds tested as modulators of GPCRs can be any small chemical compound, or a biological entity, e.g., a macromolecule such as a protein, sugar, nucleic acid or lipid. Alternatively, modulators can be genetically altered versions of GPCR. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

Binding Assays

The instant invention provides binding assays using recombinant cell lines claimed and described herein. Candidate or test compounds or agents which bind to GPCR and/or have a stimulatory or inhibitory effect on the activity or the expression of GPCR are identified either in assays that employ cells which express GPCR on the cell surface (cell-based assays) or in assays with isolated GPCR (cell-free assays). The various assays can employ a variety of variants of GPCR (e.g., full-length GPCR, a biologically active fragment of GPCR, or a fusion protein which includes all or a portion of GPCR). Moreover, GPCR can be derived from any suitable mammalian species (e.g., human GPCR, rat GPCR or murine GPCR). The assay can be a binding assay entailing direct or indirect measurement of the binding of a test compound or a known GPCR ligand to GPCR. The assay can also be an activity assay entailing direct or indirect measurement of the activity of GPCR.

In one embodiment, the invention provides assays for screening candidate or test compounds which bind to or modulate the activity of a membrane-bound (cell surface expressed) form of GPCR. Such assays can employ full-length GPCR, a biologically active fragment of GPCR, or a fusion protein which includes all or a portion of GPCR. Such test compounds can be obtained by any suitable means, e.g., from conventional compound libraries. Determining the ability of the test compound to bind to a membrane-bound form of GPCR can be accomplished, for example, by coupling the test compound with a radioisotope or enzymatic label such that binding of the test compound to the GPCR expressing cell can be measured by detecting the labeled compound in a complex. For example, the test compound can be labeled with ¹²⁵I, ³⁵S, ¹⁴C, or ³H, either directly or indirectly, and the radioisotope detected by -direct counting of radio-emission or by scintillation counting. Alternatively, the test compound can be enzymatically labeled with, for example, horseradish peroxidase, alkaline phosphatase, or luciferase, and the enzymatic label detected by determination of conversion of an appropriate substrate to product.

Binding assays can also be used to detect receptor-mediated G-protein activation (see, e.g., “Regulation of G Protein-Coupled Receptor Function and Expression” ed. Benovic, J. L. pp 119 132., 2000, Wiley-Liss, New York). Such assays include receptor-stimulated GTP Binding to G.alpha. subunits. Activation of GPCR results in GDP-GTP exchange in the G.alpha. subunit, and this exchange can be quantified and used as a direct measurement of receptor-G protein interaction. This typically involves the use of radiolabelled guanine nucleotide with the receptor either in cell free membrane preparations or artificial lipid membranes. The amount of radiolabel incorporated is used as a measure of the extent of G protein activation.

Receptor-G-protein interactions can also be examined. For example, in the absence of GTP, an activator will lead to the formation of a tight complex of a G protein (all three subunits) with the receptor. This complex can be detected in a variety of ways, as noted above. Such an assay can be modified to search for inhibitors. Adding an activator to the receptor and G protein in the absence of GTP, can be used to screen for inhibitors through measurements of the dissociation constants of the receptor-G protein complex. In the presence of GTP, release of the alpha subunit of the G protein from the other two G protein subunits serves as a criterion of activation.

Ligand binding to GPCR, a domain of a GPCR protein, or a chimeric protein can be tested in solution, in a bilayer membrane, attached to a solid phase, in a lipid monolayer, or in vesicles. Binding of a modulator can be tested using, e.g., changes in spectroscopic characteristics (e.g., fluorescence, absorbance, refractive index) hydrodynamic (e.g., shape), chromatographic, or solubility properties, as well as other techniques known in the art.

Other useful binding assays utilize changes in intrinsic tryptophan fluorescence of protein subunits. The intrinsic fluorescence of tryptophan residues undergoes an enhancement during GDP-GTP exchange. Such an enhancement can be detected using methods known in the art.

The assay can also be an expression assay entailing direct or indirect measurement of the expression of GPCR mRNA or GPCR protein. The various screening assays are combined with an in vivo assay entailing measuring the effect of the test compound on the symptoms of hematological and cardiovascular diseases, disorders of the peripheral and central nervous system, COPD, asthma, genito-urological disorders and inflammation diseases.

In a competitive binding format, binding assays comprise contacting GPCR expressing cell with a known compound which binds to GPCR to form an assay mixture, contacting the assay mixture with a test compound, and determining the ability of the test compound to interact with the GPCR expressing cell, wherein determining the ability of the test compound to interact with the GPCR expressing cell comprises determining the ability of the test compound to preferentially bind the GPCR expressing cell as compared to the known compound.

In another embodiment, the assay is a cell-based assay comprising contacting a cell expressing a membrane-bound form of GPCR (e.g., full-length GPCR, a biologically active fragment of GPCR, or a fusion protein which includes all or a portion of GPCR) expressed on the cell surface with a test compound and determining the ability of the test compound to modulate (e.g., stimulate or inhibit) the activity of the membrane-bound form of GPCR. Determining the ability of the test compound to modulate the activity of the membrane-bound form of GPCR can be accomplished by any method suitable for measuring the activity of a G-protein coupled receptor or other seven-transmembrane receptors. The activity of a seven-transmembrane receptors can be measured in a number of ways, not all of which are suitable for any given receptor. Among the measures of activity are: alteration in intracellular Ca.sup.2+ concentration, activation of phospholipase C, alteration in intracellular inositol triphosphate QP3) concentration, alteration in intracellular diacylglycerol (DAG) concentration, and alteration in intracellular adenosine cyclic 3′,5′-monophosphate (cAMP) concentration.

The cell-free assays of the present invention are amenable to use with either a membrane-bound form of a GPCR or a soluble fragment thereof. In the case of cell-free assays comprising the membrane-bound form of the polypeptide, it may be desirable to utilize a solubilizing agent such that the membrane-bound form of the polypeptide is maintained in solution. Examples of such solubilizing agents include but are not limited to non-ionic detergents such as n-octylglucoside, n-dodecyl-glucoside, n-dodecylmaltoside, octanoyl-N-methylglucamide, decanoyl-N-methyl-glucamide, Triton X-100, Triton X-114, Thesit, Isotridecypoly(ethylene glycol ether)n, 3-[(3-cholamidopropyl)dimethylamminio]-1-propane sulfonate (CRAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In some embodiments of the assays used in accordance with the present invention, it may be desirable to immobilize GPCR (or a GPCR target molecule) to facilitate separation of complexed from uncomplexed forms of one or both of the proteins, as well as to accommodate automation of the assay. Binding of a test compound to GPCR, or interaction of GPCR with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase (GST) fusion proteins or glutathione-S-transferase fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or GPCR, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of GPCR can be determined using standard techniques.

In binding assays, either the test compound or the GPCR polypeptide can comprise a detectable label, such as a fluorescent, radioisotopic, chemiluminescent, or enzymatic-label, such as horseradish peroxidase, alkaline phosphatase, or luciferase. Detection of a test compound which is bound to GPCR polypeptide can then be accomplished, for example, by direct counting of radioemmission, by scintillation counting, or by determining conversion of an appropriate substrate to a detectable product. Alternatively, binding of a test compound to a GPCR polypeptide can be determined without labeling either of the interactants. For example, a microphysiometer can be used to detect binding of a test compound with a GPCR polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and GPCR [Haseloff, (1988)].

In another embodiment of the invention, a GPCR-like polypeptide can be used as a “bait protein” in a two-hybrid assay or three-hybrid assay [Szabo, (1995); U.S. Pat. No. 5,283,317), to identify other proteins which bind to or interact with GPCR and modulate its activity. Methods for detecting such complexes, in addition to those described above for the GST-immobilized complexes, include immunodetection of complexes using antibodies which specifically bind to GPCR polypeptide or test compound, enzyme-linked assays which rely on detecting an activity of GPCR polypeptide, and SDS gel electrophoresis under non-reducing conditions.

### Functional Assays

In one aspect, the invention provides methods for producing functional assay cell lines. These methods include producing cell lines expressing GPCR proteins encoded for by nucleotide sequences selected from SEQ ID NO: 2, 4, 6, 8, 10, 12, 14, 16, and 18. A functional reporter is coupled to the binding of a ligand to the GPCR protein, such that a binding event between said ligand and said GPCR protein is detectable as a reporter activity readout. In a preferred embodiment of the invention, the cell line expressing GPCR proteins is expressing at levels of at least 150,000 copies of GPCR protein per cell.

In one embodiment, the reporter readout that is detectable upon binding of a ligand to the GPCR protein includes detection of second messenger activity. In a preferred embodiment, the second messenger activity includes a change in intracellular calcium levels, cAMP activity, and/or NFAT or CRE driven beta-lactamase.

In another embodiment, the reporter activity readout includes detection of GFP, luciferase, and of a radio-labeled molecule.

In a second aspect, the invention provides a method of using a GPCR-expressing cell line to identify a test compound which modulates activity of said GPCR. In this method, second messenger activity is measured in a cell line in the absence of said test compound—this constitutes the first measurement. A second measurement is made of second messenger activity in the presence of a test compound. A comparison of the first and second measurement that shows that there is a difference between the first measurement and the second measurement identifies the test compound as an agent modulates the activity of the GPCR. In accordance with the invention, the cell line expresses at least 150,000 copies of the GPCR protein per cell.

In a preferred embodiment, the second messenger activity measured to determine if a test compound modulates GPCR activity includes a change in intracellular calcium and a change in intracellular cAMP levels. In another preferred embodiment, the detection of second messenger activity includes high throughput screening methods.

In one embodiment of the invention, an affinity tag is attached to a GPCR protein, allowing for detection of the protein when expressed on the cellular membrane. With such an affinity tag, fluorescence-activated cell sorting (FACS) can be used for both detection as well as to quantify protein expression. In a further embodiment of the invention, FACS screening makes use of an anti-tag monoclonal antibody for both detection and quantification. In another embodiment of the invention, recombinant receptors are similarly analyzed using radio-labeled ligands combined with binding assays.

Flow cytometry is a method that can be utilized to detect surface expression of GPCRs. In traditional flow cytometry, it is common to analyze very large numbers of eukaryotic cells in a short period of time. Newly developed flow cytometers can analyze and sort up to 20,000 cells per second. In a typical flow cytometer, individual particles pass through an illumination zone and appropriate detectors, gated electronically, measure the magnitude of a pulse representing the extent of light scattered. The magnitude of these pulses are sorted electronically into “bins” or “channels”, permitting the display of histograms of the number of cells possessing a certain quantitative property as a function of channel number (Davey and Kell, 1996). It has been shown that the data accruing from flow cytometric measurements can be analyzed (electronically) rapidly enough that electronic cell-sorting procedures could be used to sort cells with desired properties into separate “buckets”, a procedure usually known as fluorescence-activated cell sorting (Davey and Kell, 1996).

Fluorescence-activated cell sorting (FACS) is often used in studies of human and animal cell lines and the control of cell culture processes. Fluorophore labeling of cells and measurement of the fluorescence can provide quantitative data about specific target molecules or subcellular components and their distribution in the cell population. Flow cytometry can quantitate virtually any cell-associated property or cell organelle for which there is a fluorescent probe (or natural fluorescence). The parameters which can be measured have previously been of particular interest in animal cell culture.

FACS machines have been employed in the present invention to analyze the success of various expression vectors and recombinant cell lines in producing high levels of GPCR proteins. Detection and counting capabilities of the FACS system are also encompassed in the methods of the present invention.

### Measuring Intracellular Calcium Levels

In one embodiment of the invention, measurement of intracellular calcium levels provides an indication of second messenger activity. Methods of measuring intracellular calcium are known to those of skill in the art. For instance, a commonly used technique is the expression of receptors of interest in Xenopus laevis oocytes followed by measurement of calcium activated chloride currents (see Weber, 1999, Biochim Biophys Acta 1421:213 233). In addition, several calcium sensitive dyes are available for the measurement of intracellular calcium. Such dyes can be membrane permeant or non-membrane permeant. Examples of useful membrane permeant dyes include acetoxymethyl ester forms of dyes that can be cleaved by intracellular esterases to form a free acid, which is no longer membrane permeant and remains trapped inside a cell. Dyes that are non-membrane permeant can be introduced into the cell by microinjection, chemical permeabilization, scrape loading and similar techniques (Haughland, 1993, in “Fluorescent and Luminescent Probes for Biological Activity” ed. Mason, W. T. pp 34 43; Academic Press, London; Haughland, 1996, in “Handbook of Fluorescent Probes and Research Chemicals”, sixth edition, Molecular Probes, Eugene, Oreg.).

Included in the present invention are assays designed to directly measure levels of cAMP produced upon modulation of adenylate cyclase activity by GPCRs. Such assays are based on the competition between endogenous cAMP and exogenously added biotinylated cAMP. The capture of cAMP is achieved by using a specific antibody conjugated to a solid material such as capture beads. Such assays are efficient at measuring both agonist and antagonist activities.

Other assays can involve determining the activity of receptors which, when activated, result in a change in the level of intracellular cyclic nucleotides, e.g., cAMP or cGMP, by activating or inhibiting downstream effectors such as adenylate cyclase. There are cyclic nucleotide-gated ion channels, e.g., rod photoreceptor cell channels and olfactory neuron channels that are permeable to cations upon activation by binding of cAMP or cGMP (see, e.g., Altenhofen et al., Proc. Natl. Acad. Sci. U.S.A. 88:9868-9872 (1991) and Dhallan et al., Nature 347:184-187 (1990)). In cases where activation of the receptor results in a decrease in cyclic nucleotide levels, it may be preferable to expose the cells to agents that increase intracellular cyclic nucleotide levels, e.g., forskolin, prior to adding a receptor-activating compound to the cells in the assay. Cells for this type of assay can be made by co-transfection of a host cell with DNA encoding a cyclic nucleotide-gated ion channel, GPCR phosphatase and DNA encoding a receptor (e.g., certain glutamate receptors, muscarinic acetylcholine receptors, dopamine receptors, serotonin receptors, and the like), which, when activated, causes a change in cyclic nucleotide levels in the cytoplasm. In one embodiment, changes in intracellular cAMP or cGMP can be measured using immunoassays.

Other screening techniques include the use of cells which express GPCR (for example, transfected CHO cells) in a system which measures extracellular pH changes caused by receptor activation [Iwabuchi, (1993)].

Functional Assay Panels

In one aspect, the present invention provides a series of functional assay panels for use in screening for modulators of GPCRs for various applications, such as for therapeutics for treatment and diagnosis of illnesses associated with GPCR activity.

The functional assay panels of the present invention are based on stable cell lines expressing different classes of GPCR. These cell lines are used as a source of representative targets for surveying drug candidate specificity in functional assays such as calcium influx. These assay panels typically include forty to fifty different GPCR-expressing cell lines, but can include upwards of 300 cell lines. In addition, these panels can be customized to screen particular compounds against GPCR expressing cell lines, such as those cell lines expressing GPCRs known to be involved in certain illnesses.

In one embodiment, samples are assigned a relative GPCR activity value of 100. Inhibition of GPCR is achieved when the GPCR activity value relative to control is about 90%, optionally 50%, optionally 25-0%. Activation of an GPCR is achieved when the GPCR activity value relative to the control is 110%, optionally 150%, 200-500%, or 1000-2000%.

Assays for mRNA

In one embodiment of the invention, transcription levels can be measured to assess the effects of a test compound on signal transduction. A host cell containing the protein of interest is contacted with a test compound for a sufficient time to effect any interactions, and then the level of gene expression is measured. The amount of time to effect such interactions may be empirically determined, such as by measuring the level of transcription as a function of time. The amount of transcription may be measured by using methods known to those of skill in the art. For example, mRNA expression of the protein of interest may be detected using Northern blots or their polypeptide products may be identified using immunoassays. Alternatively, transcription based assays using reporter gene may be used as described in U.S. Pat. No. 5,436,128, herein incorporated by reference. The reporter genes can be, e.g., chloramphenicol acetyltransferase, firefly luciferase, bacterial luciferase, .beta.-galactosidase and alkaline phosphatase. Furthermore, the protein of interest can be used as an indirect reporter via attachment to a second reporter such as green fluorescent protein (see, e.g., Mistili & Spector, Nature Biotechnology 15:961-964 (1997)).

The amount of transcription is then compared to the amount of transcription in either the same cell in the absence of the test compound, or it may be compared with the amount of transcription in a substantially identical cell that lacks the protein of interest. A substantially identical cell may be derived from the same cells from which the recombinant cell was prepared but which had not been modified by introduction of heterologous DNA.

### Solid State and Soluble High Throughput Assays

In one embodiment the invention provides soluble assays using molecules corresponding to GPCR protein domains, such as ligand binding domain, an extracellular domain, a transmembrane domain (e.g., one comprising seven transmembrane regions and cytosolic loops), and a cytoplasmic domain, an active site, a subunit association region, etc. Such domains may be covalently linked to a heterologous protein to create a chimeric molecule. In another embodiment, the invention provides solid phase based in vitro assays in a high throughput format, where the domain, chimeric molecule, GPCR, or cell or tissue expressing an GPCR is attached to a solid phase substrate.

In certain high throughput assays, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators.

The molecule of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest (e.g., the signal transduction molecule of interest) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of antibodies are commercially available and described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

### Treatment of Disease

In one aspect, the invention provides a method of treating a condition associated with a GPCR protein. Such a method involves administering to a subject in need of such treatment an effective amount of an antibody that specifically binds to a structural feature of a GPCR protein. In a preferred embodiment of the invention, the antibody is raised against a cell line expressing at least 150,000 copies of a GPCR protein per cell.

In one embodiment of the invention, the treatment involves administering an effective amount of an antibody that is conjugated to a therapeutic entity. In a preferred embodiment of the invention, the antibody is conjugated to an anti-cancer therapeutic entity.

In one embodiment of the invention, the condition requiring treatment that is associated with a GPCR protein is neoplastic growth.

In another embodiment of the invention, the condition requiring treatment that is associated with a GPCR protein is breast cancer.

In still another embodiment, an element or symptom of the condition requiring treatment that is associated with a GPCR protein is inflammation.

### Drug Discovery

In one aspect, the invention provides methods for targeted drug discovery and pharmaceutical design based on secondary and tertiary structures of GPCR proteins. In one embodiment of the invention, structural information on a GPCR protein is obtained from a study of proteins isolated from cells expressing at least 150,000 copies of the GPCR protein per cell.

Modulators of GPCR activity may be tested using GPCR polypeptides. The polypeptide can be isolated, expressed in a cell, expressed in a membrane derived from a cell, expressed in tissue or in an animal, either recombinant or naturally occurring. For example, breast cancer cells, normal prostate epithelial cells, placenta, testis tissue, transformed cells, or membranes can be used. Signal transduction can also be examined in vitro with soluble or solid state reactions, or by using a chimeric molecule such as an extracellular domain of a receptor covalently linked to a heterologous signal transduction domain, or with a heterologous extracellular domain covalently linked to the transmembrane and or cytoplasmic domain of a receptor. Gene amplification can also be examined. Furthermore, ligand-binding domains of the protein of interest can be used in vitro in soluble or solid state reactions to assay for ligand binding.

Test compounds can be tested for the ability to increase or decrease GPCR activity of a GPCR polypeptide. The GPCR activity can be measured after contacting either a purified GPCR, a cell membrane preparation, or an intact cell with a test compound. A test compound which decreases GPCR activity by at least about 10, preferably about 50, more preferably about 75, 90, or 100% is identified as a potential agent for decreasing GPCR activity. A test compound which increases GPCR activity by at least about 10, preferably about 50, more preferably about 75, 90, or 100% is identified as a potential agent for increasing GPCR activity.

### GPCR-Directed Compound Libraries

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical and biochemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No: WO 91/19735), encoded peptides (e.g., PCT Publication WO 93/20242), random bio-oligomers (e.g., PCT Publication No: WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem. Tech, Louisville Ky., Symphony, Rainin, Wobum, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Lead-finding libraries can follow molecular mimicry principles and use the substantial medicinal chemistry knowledge that has been generated during the last decade around GPCR compounds. Also useful in drug design are lead/drug likeness and computational combinatorial library design.

Studies suggest that certain classes and structural properties of ligands are important in designing drugs for particular receptors. For example, divalent ligands often selectively target opioid receptor heterodimers, while protein mimetics with β-turn/α-helix domains are important features for drugs mimicking hormones such as somatostatin and angiotensin. Cyclic α-peptides and β/γ peptides may also serve as a basis for drug design for some GPCRs.

The use of privileged substructures or molecular master keys that are target-class-specific or that mimic protein secondary structure elements is a commonly used strategy in the art. The privileged-structure approach utilizes molecular scaffolds or selected substructures able to provide high-affinity ligands for diverse receptor targets. Empirically derived privileged structures include spiropiperidines, biphenyl-tetrazoles, benzimidazoles, and benzofurans. Chemoinformatics methods may also enable the automatic identification and extraction of privileged structures, which is particularly important for developing knowledge from high throughput screening data. Software such as Scitegic Pipeline Pilot can also be used to generate a data-pipelining protocol that generates frequency analysis based on the input of different reference sets.

Thematic analysis may also be used to develop drugs for GPCR proteins. In this method, a class of proteins, such as a GPCR family, is analyzed to develop a classification based on the pairing of “sequence themes” and ligand structural motifs. A sequence theme is a consensus collection of amino acids within the central binding cavity, and a motif is a specific structural element binding to such a particular microenvironment of the binding site. This compilation of themes and motifs can then be used to generate focused discovery libraries and to increase the lead optimization efficiency for the drug targets.

Individual compound libraries that target subsets of GPCRs, such as orphan receptors, share a predefined combination of themes consisting of a central dominant theme and peripheral ancillary themes. The library scaffold can then be designed to complement the central theme, with incorporation of a variety of structural motifs that address the individual sequence themes. Such libraries, consisting of approximately 1000 compounds, can then be thought of as representing a number of predefined themes which are either present or absent in a given receptor. This “fingerprinting” approach allows a score to be assigned to a particular library of compounds as to appropriateness of that group of compounds for a particular receptor. Thematic analysis could also be used to develop new combinations of used and unused themes to increase affinity and selectivity of lead compounds in a pipeline.

Other design strategies include related computer-assisted drug design, which makes use of selected reference compound sets and molecular descriptors together with cheminformatics methods to compare and rank the similarity of designed candidate molecules. Homology-based similarity searching can also identify potential ligands for orphan receptors. Artificial neural networks, self-organizing maps, and support vector machines may also be used in the drug design process—these methods align chemical and biological spaces based on mapping procedures to determine which parts of the chemical-property space correspond to specific target-families or therapeutic activities.

### Administration and Pharmaceutical Compositions

GPCR modulators can be administered directly to the mammalian subject for modulation of signal transduction in vivo, e.g., for the treatment of a cancer such as breast cancer. Administration is by any of the routes normally used for introducing a modulator compound to the tissue to be treated. GPCR modulators can be administered in any suitable manner, optionally with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17.sup.th ed. 1985)).

GPCR modulators, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, by orally, topically, intravenously, intraperitoneally, intravesically or intrathecally. Optionally, the compositions are administered orally or nasally. The formulations of compounds can be presented in unit-dose or multi-dose sealed containers, such as ampules and vials. Solutions and suspensions can be prepared from sterile powders, granules, and tablets of the kind previously described. The modulators can also be administered as part a of prepared food or drug.

The dose administered to a patient, in the context of the present invention should be sufficient to effect a beneficial response in the subject over time. Such doses are administered prophylactically or to an individual already suffering from the disease. The compositions are administered to a patient in an amount sufficient to elicit an effective protective or therapeutic response in the patient. An amount adequate to accomplish this is defined as “therapeutically effective dose.” The dose will be determined by the efficacy of the particular GPCR modulators (e.g., GPCR antagonists and anti-GPCR antibodies) employed and the condition of the subject, as well as the body weight or surface area of the area to be treated. The size of the dose will also be determined by the existence, nature, and extent of any adverse side-effects that accompany the administration of a particular compound or vector in a particular subject.

In determining the effective amount of the modulator to be administered a physician may evaluate circulating plasma levels of the modulator, modulator toxicities, and the production of anti-modulator antibodies. In general, the dose equivalent of a modulator is from about 1 ng/kg to 10 mg/kg for a typical subject.

GPCR modulators of the present invention can be administered at a rate determined by the LD-50 of the modulator, and the side-effects of the inhibitor at various concentrations, as applied to the mass and overall health of the subject. Administration can be accomplished via single or divided doses.

### Purification of GPCRs from Recombinant Cells

Recombinant proteins are expressed by transformed bacteria or eukaryotic cells such as CHO cells or insect cells in large amounts, typically after promoter induction, but expression can be constitutive. Promoter induction with IPTG is a one example of an inducible promoter system. Cells are grown according to standard procedures in the art. Fresh or frozen cells can be used for isolation of protein.

Proteins expressed in bacteria may form insoluble aggregates (“inclusion bodies”). Several protocols are suitable for purification of GPCR inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternative methods of lysing bacteria are apparent to those of skill in the art (see, e.g., Sambrook et al., supra; Ausubel et al., supra).

If necessary, inclusion bodies are solubilized, and the lysed cell suspension is typically centrifuged to remove unwanted insoluble matter. Proteins that form inclusion bodies may be renatured by elution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. The GPCR is separated from other bacterial proteins by standard separation techniques, e.g., with Ni-NTA agarose resin.

It is also possible to purify the GPCR from bacteria periplasm. After lysis of the bacteria, when the GPCR is exported into the periplasm of the bacteria, the periplasmic fraction of the bacteria can be isolated by cold osmotic shock, as well as by other methods known to those of skill in the art. To isolate recombinant proteins from the periplasm, the bacterial cells are centrifuged to form a pellet. The pellet is resuspended in a buffer containing 20% sucrose. To lyse the cells, the bacteria are centrifuged and the resultant pellet is resuspended in ice-cold 5 mM MgSO.sub.4 and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of GPCR can be used to isolated it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed.

Column Chromatography

GPCRs can also be separated from other proteins on the basis of its size, net surface charge, hydrophobicity, and affinity for ligands using techniques of column chromatography known in the art. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Suitable test compounds for use in the screening assays of the invention can be obtained from any suitable source, e.g., conventional compound libraries. The test compounds can also be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds [Lam, (1997)]. Examples of methods for the synthesis of molecular libraries can be found in the art. Libraries of compounds may be presented in solution or on beads, bacteria, spores, plasmids or phage.

Quantification of Protein Production

Western blot (immunoblot) analysis can be used to detect and quantify the presence of -GPCR in the sample. The technique generally comprises separating sample proteins by gel electrophoresis on the basis of molecular weight, transferring the separated proteins to a suitable solid support, (such as a nitrocellulose filter, a nylon filter, or derivitized nylon filter), and incubating the sample with the antibodies that specifically bind GPCR. The anti-GPCR antibodies specifically bind to the GPCR on the solid support. These antibodies may be directly labeled or alternatively may be subsequently detected using labeled antibodies (e.g., labeled sheep anti-mouse antibodies) that specifically bind to the anti-GPCR antibodies.

Labels

The particular label or detectable group used in an assay is not a critical aspect of the invention, as long as it does not significantly interfere with the specific binding of the antibody used in the assay. The detectable group can be any material having a detectable physical or chemical property. Such detectable labels have been well-developed in the field and, in general, almost any label useful in assay methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., 3H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. The choice of label used in an assay depends on factors such as the required sensitivity, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize GPCRs, or secondary antibodies that recognize anti-GPCR.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazined-iones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

In addition, techniques developed for the production of “chimeric antibodies”, the splicing of antibody genes from different species to obtain a molecule with appropriate antigen specificity and biological activity can be used. Monoclonal and other antibodies can be “humanized” to prevent a patient from mounting an immune response against the antibody when it is used therapeutically. Such antibodies may be sufficiently similar in sequence to human antibodies to be used directly in therapy or may require alteration of a few key residues. For example, sequence differences between rodent antibodies and human sequences can be minimized by replacing residues which differ from those in the human sequences by site directed mutagenesis of individual residues or by grating of entire complementarity determining regions. Antibodies which specifically bind to GPCR can thus contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to GPCR. Antibodies with related specificity, but of distinct idiotypic composition, can be generated by chain shuffling from random combinatorial immunoglobin libraries. Single-chain antibodies also can be constructed using a DNA amplification method, such as PCR, using hybridoma cDNA as a template. Single-chain antibodies can be mono- or bispecific, and can be bivalent or tetravalent. Construction of tetravalent, bispecific single-chain antibodies is taught. A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology.

Antibodies according to the invention can be purified by methods well known in the art. For example, antibodies can be affinity purified by passage over a column to which GPCR is bound. The bound antibodies can then be eluted from the column using a buffer with a high salt concentration

### Immunoassays

In addition to the detection of GPCR genes and gene expression using nucleic acid hybridization technology, one can also use immunoassays to detect GPCRs, e.g., to identify cells such as cancer cells, in particular breast cancer cells, and variants of GPCRs. Immunoassays can be used to qualitatively or quantitatively analyze GPCRs. A general overview of the applicable technology can be found in Harlow & Lane, Antibodies: A Laboratory Manual (1988).

Immunoassays use a labeling agent to specifically bind to and label the complex formed by the antibody and antigen. The labeling agent may itself be one of the moieties comprising the antibody/antigen complex. Alternatively, the labeling agent may be a third moiety, such a secondary antibody that specifically binds to the antibody/GPCR complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 111:1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Noncompetitive immunoassays are assays in which the amount of antigen is directly measured. In one preferred “sandwich” assay, for example, the anti-GPCR antibodies can be bound directly to a solid substrate on which they are immobilized. These immobilized antibodies then capture GPCRs present in the test sample. The GPCR thus immobilized is then bound by a labeling agent, such as a second GPCR antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

In competitive assays, the amount of GPCR present in the sample is measured indirectly by measuring the amount of a known, added (exogenous) GPCR displaced (competed away) from an anti-GPCR antibody by the unknown GPCR present in a sample. In one competitive assay, a known amount of GPCR is added to a sample and the sample is then contacted with an antibody that specifically binds to the GPCR. The amount of exogenous GPCR bound to the antibody is inversely proportional to the concentration of GPCR present in the sample. In a particularly preferred embodiment, the antibody is immobilized on a solid substrate. The amount of GPCR bound to the antibody may be determined either by measuring the amount of GPCR present in a GPCR/antibody complex, or alternatively by measuring the amount of remaining uncomplexed protein. The amount of GPCR may be detected by providing a labeled GPCR molecule.

A hapten inhibition assay is another preferred competitive assay. In this assay the known GPCR is immobilized on a solid substrate. A known amount of anti-GPCR antibody is added to the sample, and the sample is then contacted with the immobilized GPCR. The amount of anti-GPCR antibody bound to the known immobilized GPCR is inversely proportional to the amount of GPCR present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Immunoassays in the competitive binding format can also be used for crossreactivity determinations. For example, a protein at least partially encoded by SEQ ID NOS: 1, 3, 5, 7, 9, 11, 13, 15, or 17 can be immobilized to a solid support. Proteins (e.g., GPCR proteins and homologs) are added to the assay that compete for binding of the antisera to the immobilized antigen. The ability of the added proteins to compete for binding of the antisera to the immobilized protein is compared to the ability of GPCRs encoded by SEQ ID NO: 1, 3, 5, 7, 9, 11, 13, 15, or 17 to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of an GPCR, to the immunogen protein (i.e., the GPCR of SEQ ID NOS: 1, 3, 5, 7, 9, 11, 13, 15, or 17). In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein encoded by SEQ ID NOS: 1, 3, 5, 7, 9, 11, 13, 15, or 17 that is required to inhibit 50% of binding, then the second protein is said to specifically bind to the polyclonal antibodies generated to a GPCR immunogen.

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Other assay formats include liposome immunoassays (LIA), which use liposomes designed to bind specific molecules (e.g., antibodies) and release encapsulated reagents or markers upon a binding event. The released chemicals are then detected according to standard techniques (see Monroe et al., Amer. Clin. Prod. Rev. 5:34-41 (1986)).

### Deorphanization

Among known GPCR proteins some 200 exist for which their natural ligand is unknown. These “orphan” GPCRs have the potential to serve as targets for novel drugs and novel indications. Yet, to be used in pharmaceutical research, these GPCRs need first to be “deorphanized”, i.e. their natural ligands need to be discovered to allow for development of applications such as high-throughput screening assays.

Deorphanization refers to the identification of activating ligands is a key task in reverse molecular pharmacology. Identifying receptor-agonist pairs usually allows the rapid elucidation of the physiological role of both partners, sometimes putting them in unexpected contexts. Although bioinformatics methods are initially helpful to successfully direct ligand-pairing experiments, deorphanization strategies generally rely on biological screening of orphan GPCRs expressed in recombinant expression systems such as immortalized mammalian cells, yeast, and Xenopus melanophores.

Agonist ligand libraries used for deorphanization can include small molecules, peptides, proteins, lipids or tissue extracts. Identification of an activating agonistic ligand of the cell-surface-expressed receptors is often dependent on the activation of an intracellular signaling cascade.

A difficulty in assay design for orphan GPCRs is that the signaling cascade is not known for a new orphan receptor. Therefore, generic assay systems amenable for high throughput screening are generally used to screen large surrogate ligand collections. One successful approach for deorphanization uses fluorescent imaging plate reader (FLIPR) screening technology, which detects ligand-induced intracellular Ca⁺² mobilization.

Given the broad chemical diversity of the molecules that are recognized by GPCRs, deorphanization libraries try to cover as many known active chemical classes as possible. The term “surrogate agonist library” is also appropriate given that the purpose of these libraries is to find a chemical compound that selectively activates a given orphan receptor of interest. Typically, compounds identical or similar to previously identified GPCR agonists are included together with approved drugs and other reference compounds with known bioactivity, such as primary metabolites like the KEGG compound set, or commercially available compilations like the Tocris LOPAC, the Prestwick, or the Sial Biomol sets.

Typically, the size of deorphanization or surrogate libraries is on the order of a few thousand well-characterized compounds amenable for medium-throughput screening. The design of lead-finding libraries follows the same molecular mimicry principles and makes best use of the substantial medicinal chemistry knowledge generated during the last decades around GPCR compounds together with more modern concepts, including lead/drug likeness and computational combinatorial library design. Focused library design concepts target the classical binding sites in general, while design concepts of bivalent ligands and allosteric ligands can be used as the understanding of the GPCR oligomerization phenomenon increases.

### Diseases Associated with GPCRs

Mutations in GPCRs have been associated with a wide variety of illnesses, particularly cancers and diseases involving inflammation.

Some disease-causing mutations result in constitutive receptor activation, such as in Jansen's disease, where the hypercalcemia and skeletal dysplasia found in many cases is the result of a constitutively overactive parathyroid hormone/parathyroid hormone related protein receptor. In such diseases, inhibitors of activation are of particular interest as potential therapeutics.

Virally encoded GPCRs may also have a direct role in human diseases. For example, Kaposi's sarcoma-associated herpes virus has been implicated in Kaposi's sarcomagenesis, and the human cytomegalovirus-encoded GPCRs have been implicated in atherosclerosis.

Certain GPCRs are associated with the disorders of the peripheral and central nervous system (CNS), cardiovascular diseases, hematological diseases, cancer, inflammation, urological diseases, respiratory diseases and gastroenterological diseases. Such disorders may include a wide range of diseases, as discussed further below.

Nervous System Disorders

CNS disorders include disorders of the central nervous system as well as disorders of the peripheral nervous system.

CNS disorders include, but are not limited to brain injuries, cerebrovascular diseases and their consequences, Parkinson's disease, corticobasal degeneration, motor neuron disease, dementia, including ALS, multiple sclerosis, traumatic brain injury, stroke, post-stroke, post-traumatic brain injury, and small-vessel cerebrovascular disease. Dementias, such as Alzheimer's disease, vascular dementia, dementia with Lewy bodies, frontotemporal dementia and Parkinsonism linked to chromosome 17, frontotemporal dementias, including Pick's disease, progressive nuclear palsy, corticobasal degeneration, Huntington's disease, thalamic degeneration, Creutzfeld-Jakob dementia, HIV dementia, schizophrenia with dementia, and Korsakoff's psychosis, within the meaning of the definition are also considered to be CNS disorders. Jakob Similarly, cognitive-related disorders, such as mild cognitive impairment, age-associated memory impairment, age-related cognitive decline, vascular cognitive impairment, attention deficit disorders, attention deficit hyperactivity disorders, and memory disturbances in children with learning disabilities are also considered to be CNS disorders. Jakob Pain, within the meaning of this definition, is also considered to be a CNS disorder. Pain can be associated with CNS disorders, such as multiple sclerosis, spinal cord injury, sciatica, failed back surgery syndrome, traumatic brain injury, epilepsy, Parkinson's disease, post-stroke, and vascular lesions in the brain and spinal cord (e.g., infarct, hemorrhage, vascular malformation). Non-central neuropathic pain includes that associated with post mastectomy pain, phantom feeling, reflex sympathetic dystrophy (RSD), trigeminal neuralgiaradioculopathy, post-surgical pain, HIV/AIDS related pain, cancer pain, metabolic neuropathies (e.g., diabetic neuropathy, vasculitic neuropathy secondary to connective tissue disease), paraneoplastic polyneuropathy associated, for example, with carcinoma of lung, or leukemia, or lymphoma, or carcinoma of prostate, colon or stomach, trigeminal neuralgia, cranial neuralgias, and post-herpetic neuralgia. Pain associated with peripheral nerve damage, central pain (i.e. due to cerebral ischemia) and various chronic pain i.e., lumbago, back pain (low back pain), inflammatory and/or rheumatic pain. Headache pain (for example, migraine with aura, migraine without aura, and other migraine disorders), episodic and chronic tension-type headache, tension-type like headache, cluster headache, and chronic paroxysmal hemicrania are also CNS disorders. Jakob Visceral pain such as pancreatits, intestinal cystitis, dysmenorrhea, irritable Bowel syndrome, Crohn's disease, biliary colic, ureteral colic, myocardial infarction and pain syndromes of the pelvic cavity, e.g., vulvodynia, orchialgia, urethral syndrome and protatodynia are also CNS disorders. Jakob Also considered to be a disorder of the nervous system are acute pain, for example postoperative pain, and pain after trauma. Jakob The human GPCR is highly expressed in the following brain tissues: brain, Alzheimer brain, cerebellum (right), cerebellum (left), cerebral cortex, Alzheimer brain frontal lobe, occipital lobe, pons, substantia nigra, cerebral meninges, corpus callosum, dorsal root ganglia, neuroblastoma IMR32 cells. The expression in brain tissues and in particular the differential expression between diseased tissue Alzheimer brain and healthy tissue brain, between diseased tissue Alzheimer brain frontal lobe and healthy tissue frontal lobe demonstrates that the human GPCR or mRNA can be utilized to diagnose nervous system diseases. Additionally the activity of the human GPCR can be modulated to treat nervous system diseases.

Cardiovascular Disorders

GPCRs are highly expressed in the following cardiovascular related tissues: heart, pericardium, heart atrium (right), heart atrium (left), artery, coronary artery, coronary artery sclerotic. Expression in the above mentioned tissues and in particular the differential expression between diseased tissue coronary artery sclerotic and healthy tissue coronary artery indicates that GPCR protein, DNA or mRNA can be utilized to diagnose cardiovascular diseases.

Heart failure is defined as a pathophysiological state in which an abnormality of cardiac function is responsible for the failure of the heart to pump blood at a rate commensurate with the requirement of the metabolizing tissue. It includes all forms of pumping failures such as high-output and low-output, acute and chronic, right-sided or left-sided, systolic or diastolic, independent of the underlying cause.

Myocardial infarction (MI) is generally caused by an abrupt decrease in coronary blood flow that follows a thrombotic occlusion of a coronary artery previously narrowed by arteriosclerosis. MI prophylaxis (primary and secondary prevention) is included as well as the acute treatment of MI and the prevention of complications. Ischemic diseases are conditions in which the coronary flow is restricted resulting in a perfusion which is inadequate to meet the myocardial requirement for oxygen. This group of diseases includes stable angina, unstable angina and asymptomatic ischemia.

Arrhythmias include all forms of atrial and ventricular tachyarrhythmas, atrial tachycardia, atrial flutter, atrial fibrillation, atrio-ventricular reentrant tachycardia, preexitation syndrome, ventricular tachycardia, ventricular flutter, ventricular fibrillation, as well as bradycardic forms of arrhythmias.

Hypertensive vascular diseases include primary as well as all kinds of secondary arterial hypertension, renal, endocrine, neurogenic, others. The genes may be used as drug targets for the treatment of hypertension as well as for the prevention of all complications arising from cardiovascular diseases.

Peripheral vascular diseases are defined as vascular diseases in which arterial and/or venous flow is reduced resulting in an imbalance between blood supply and tissue oxygen demand. It includes chronic peripheral arterial occlusive disease (PAOD), acute arterial thrombosis and embolism, inflammatory vascular disorders, Raynaud's phenomenon and venous disorders.

Cardiovascular diseases include but are not limited to disorders of the heart and the vascular system like congestive heart failure, myocardial infarction, ischemic diseases of the heart, all kinds of atrial and ventricular arrhythmias, hypertensive vascular diseases, peripheral vascular diseases, and atherosclerosis.

Examples of disorders of lipid metabolism are hyperlipidemia (abnormally high levels of fats (cholesterol, triglycerides, or both) in the blood, may be caused by family history of hyperlipidemia), obesity, a high-fat diet, lack of exercise, moderate to high alcohol consumption, cigarette smoking, poorly controlled diabetes, and an underactive thyroid gland), hereditary hyperlipidemias (type I hyperlipoproteinemia (familial hyperchylomicronemia), type II hyperlipoproteinemia (familial hypercholesterolemia), type In hyperlipoproteinemia, type IV hyperlipoproteinemia, or type V hyperlipoproteinemia), hypolipoproteinemia, lipidoses (caused by abnormalities in the enzymes that metabolize fats), Gaucher's disease, Niemann-Pick disease, Fabry's disease, Wolman's disease, cerebrotendinous xanthomatosis, sitosterolemia, Refsum's disease, or Tay-Sachs disease.

Kidney Disorders

Kidney disorders may lead to hypertension or hypotension. Examples of kidney problems possibly leading to hypertension are renal artery stenosis, pyelonephritis, glomerulonephritis, kidney tumors, polycistic kidney disease, injury to the kidney, or radiation therapy affecting the kidney. Excessive urination may lead to hypotension.

Hematological Disorders

Hematological disorders comprise diseases of the blood and all its constituents as well as diseases of organs and tissues involved in the generation or degradation of all the constituents of the blood. They include but are not limited to 1) Anemias, 2) Myeloproliferative Disorders, 3) Hemorrhagic Disorders, 4) Leukopenia, 5) Eosinophilic Disorders, 6) Leukemias, 7) Lymphomas, 8) Plasma Cell Dyscrasias, 9) Disorders of the Spleen in the course of hematological disorders. Disorders according to 1) include, but are not limited to anemias due to defective or deficient hem synthesis, deficient erythropoiesis. Disorders according to 2) include, but are not limited to polycythemia vera, tumor-associated erythrocytosis, myelofibrosis, thrombocythemia. Disorders according to 3) include, but are not limited to vasculitis, thrombocytopenia, heparin-induced thrombocytopenia, thrombotic thrombocytopenic purpura, hemolytic-uremic syndrome, hereditary and acquired disorders of platelet function, hereditary coagulation disorders. Disorders according to 4) include, but are not limited to neutropenia, lymphocytopenia. Disorders according to 5) include, but are not limited to hypereosinophilia, idiopathic hypereosinophilic syndrome. Disorders according to 6) include, but are not limited to acute myeloic leukemia, acute lymphoblastic leukemia, chronic myelocytic leukemia, chronic lymphocytic leukemia, myelodysplastic syndrome. Disorders according to 7) include, but are not limited to Hodgkin's disease, non-Hodgkin's lymphoma, Burkitt's lymphoma, mycosis fungoides cutaneous T-cell lymphoma. Disorders according to 8) include, but are not limited to multiple myeloma, macroglobulinemia, heavy chain diseases. In extension of the preceding idiopathic thrombocytopenic purpura, iron deficiency anemia, megaloblastic anemia (vitamin B12 deficiency), aplastic anemia, thalassemia, malignant lymphoma bone marrow invasion, malignant lymphoma skin invasion, hemolytic uremic syndrome, giant platelet disease are considered to be hematological diseases too.

The human GPCR is highly expressed in the following tissues of the hematological system: leukocytes peripheral blood), bone marrow, erythrocytes, lymphnode, thymus, thrombocytes, bone marrow CD34+ cells, bone marrow CD15+ cells, spleen, spleen liver cirrhosis.

Gastrointestinal and Liver Diseases

Gastrointestinal diseases comprise primary or secondary, acute or chronic diseases of the organs of the gastrointestinal tract which may be acquired or inherited, benign or malignant or metaplastic, and which may affect the organs of the gastrointestinal tract or the body as a whole. They comprise but are not limited to 1) disorders of the esophagus like achalasia, vigoruos achalasia, dysphagia, cricopharyngeal incoordination, pre-esophageal dysphagia, diffuse esophageal spasm, globus sensation, Barrett's metaplasia, gastroesophageal reflux, 2) disorders of the stomach and duodenum like functional dyspepsia, inflammation of the gastric mucosa, gastritis, stress gastritis, chronic erosive gastritis, atrophy of gastric glands, metaplasia of gastric tissues, gastric ulcers, duodenal ulcers, neoplasms of the stomach, 3) disorders of the pancreas like acute or chronic pancreatitis, insufficiency of the exocrinic or endocrinic tissues of the pancreas like steatorrhea, diabetes, neoplasms of the exocrine or endocrine pancreas like 3.1) multiple endocrine neoplasia syndrome, ductal adenocarcinoma, cystadenocarcinoma, islet cell tumors, insulinoma, gastrinoma, carcinoid tumors, glucagonoma, Zollinger-Ellison syndrome, Vipoma syndrome, malabsorption syndrome, 4) disorders of the bowel like chronic inflammatory diseases of the bowel, Crohn's disease, ileus, diarrhea and constipation, colonic inertia, megacblon, malabsorption syndrome, ulcerative colitis, 4.1) functional bowel disorders like irritable bowel syndrome, 4.2) neoplasms of the bowel like familial polyposis, adenocarcinoma, primary malignant lymphoma, carcinoid tumors, Kaposi's sarcoma, polyps, cancer of the colon and rectum.

Liver diseases comprise primary or secondary, acute or chronic diseases or injury of the liver which may be acquired or inherited, benign or malignant, and which may affect the liver or the body as a whole. They comprise but are not limited to disorders of the bilirubin metabolism, jaundice, syndroms of Gilbert's, Crigler-Najjar, Dubin-Johnson and Rotor; intrahepatic cholestasis, hepatomegaly, portal hypertension, ascites, Budd-Chiari syndrome, portal-systemic encephalopathy, fatty liver, steatosis, Reye's syndrome, liver diseases due to alcohol, alcoholic hepatitis or cirrhosis, fibrosis and cirrhosis, fibrosis and cirrhosis of the liver due to inborn errors of metabolism or exogenous substances, storage diseases, syndromes of Gaucher's, Zellweger's, Wilson's-disease, acute or chronic hepatitis, viral hepatitis and its variants, inflammatory conditions of the liver due to viruses, bacteria, fungi, protozoa, helminths; drug induced disorders of the liver, chronic liver diseases like primary sclerosing cholangitis, alpha.sub.1-antitrypsin-deficiency, primary biliary cirrhosis, postoperative liver disorders like postoperative intrahepatic cholestasis, hepatic granulomas, vascular liver disorders associated with systemic disease, benign or malignant neoplasms of the liver, disturbance of liver metabolism in the new-born or prematurely born.

GPCRs are highly expressed in the following tissues of the gastro-enterological system: esophagus, esophagus tumor, stomach tumor, rectum. The expression in the above mentioned tissues and in particular the differential expression between diseased tissue esophagus tumor and healthy tissue esophagus, between diseased tissue stomach tumor and healthy tissue stomach demonstrates that GPCR DNA, mRNA and polypeptides can be utilized to diagnose of gastroenterological disorders. Additionally the activity of the human GPCR can be modulated to treat gastroenterological disorders.

Cancer Disorders

Cancer disorders within the scope of this definition comprise any disease of an organ or tissue in mammals characterized by poorly controlled or uncontrolled multiplication of normal or abnormal cells in that tissue and its effect on the body as a whole. Cancer diseases within the scope of the definition comprise benign neoplasms, dysplasias, hyperplasias as well as neoplasms showing metastatic growth or any other transformations like e.g. leukoplakias which often precede a breakout of cancer. Cells and tissues are cancerous when they grow more rapidly than normal cells, displacing or spreading into the surrounding healthy tissue or any other tissues of the body described as metastatic growth, assume abnormal shapes and sizes, show changes in their nucleocytoplasmatic ratio, nuclear polychromasia, and finally may cease.

Cancerous cells and tissues may affect the body as a whole when causing paraneoplastic syndromes or if cancer occurs within a vital organ or tissue, normal function will be impaired or halted, with possible fatal results. The ultimate involvement of a vital organ by cancer, either primary or metastatic, may lead to the death of the mammal affected. Cancer tends to spread, and the extent of its spread is usually related to an individual's chances of surviving the disease.

Cancers are generally said to be in one of three stages of growth: early, or localized, when a tumor is still confined to the tissue of origin, or primary site; direct extension, where cancer cells from the tumor have invaded adjacent tissue or have spread only to regional lymph nodes; or metastasis, in which cancer cells have migrated to distant parts of the body from the primary site, via the blood or lymph systems, and have established secondary sites of infection.

Cancer is said to be malignant because of its tendency to cause death if not treated. Benign tumors usually do not cause death, although they may if they interfere with a normal body function by virtue of their location, size, or paraneoplastic side effects. Hence benign tumors fall under the definition of cancer within the scope of this definition as well. In general, cancer cells divide at a higher rate than do normal cells, but the distinction between the growth of cancerous and normal tissues is not so much the rapidity of cell division in the former as it is the partial or complete loss of growth restraint in cancer cells and their failure to differentiate into a useful, limited tissue of the type that characterizes the functional equilibrium of growth of normal tissue.

The term “cancer” as referred to herein is not limited to simple benign neoplasia but comprises any other benign and malign neoplasia like 1) Carcinoma, 2) Sarcoma, 3) Carcinosarcoma, 4) Cancers of the blood-forming tissues, 5) tumors of nerve tissues including the brain, 6) cancer of skin cells.

GPCRs are expressed in the following cancer tissues: esophagus tumor, stomach tumor, lung tumor, ovary tumor, kidney tumor. The expression in the above mentioned tissues and in particular the differential expression between diseased tissue esophagus tumor and healthy tissue esophagus, between diseased tissue stomach tumor and healthy tissue stomach, between diseased tissue lung tumor and healthy tissue lung, between diseased tissue ovary tumor and healthy tissue ovary, between diseased tissue kidney tumor and healthy tissue kidney demonstrates that the human GPCR DNA, mRNA and polypeptides can be utilized to diagnose of cancer. Additionally the activity of GPCR can be modulated to treat cancer.

Inflammatory Diseases

Inflammatory diseases comprise diseases triggered by cellular or non-cellular mediators of the immune system or tissues causing the inflammation of body tissues and subsequently producing an acute or chronic inflammatory condition. Examples for such inflammatory diseases are hypersensitivity reactions of type I-IV, for example but not limited to hypersensitivity diseases of the lung including asthma, atopic diseases, allergic rhinitis or conjunctivitis, angioedema of the lids, hereditary angioedema, antireceptor hypersensitivity reactions and autoimmune diseases, Hashimoto's thyroiditis, systemic lupus erythematosus, Goodpasture's syndrome, pemphigus, myasthenia gravis, Grave's and Raynaud's disease, type B insulin-resistant diabetes, rheumatoid arthritis, psoriasis, Crohn's disease, scleroderma, mixed connective tissue disease, polymyositis, sarcoidosis, glomerulonephritis, acute or chronic host versus graft reactions.

GPCR is expressed in the following tissues of the immune system and tissues responsive to components of the immune system as well as in the following tissues responsive to mediators of inflammation: leukocytes (peripheral blood), bone marrow, bone marrow CD15+ cells, spleen liver cirrhosis, lung COPD. The expression in the above mentioned tissues and in particular the differential expression between diseased tissue spleen liver cirrhosis and healthy tissue spleen, between diseased tissue lung COPD and healthy tissue lung demonstrates that GPCR DNA, mRNA and polypeptides can be utilized to diagnose of inflammatory diseases. Additionally the activity of GPCR can be modulated to treat inflammatory diseases.

Disorders Related to Pulmology

Asthma is thought to arise as a result of interactions between multiple genetic and environmental factors and is characterized by three major features: 1) intermittent and reversible airway obstruction caused by bronchoconstriction, increased mucus production, and thickening of the walls of the airways that leads to a narrowing of the airways, 2) airway hyperresponsiveness, and 3) airway inflammation. Certain cells are critical to the inflammatory reaction of asthma and they include T cells and antigen presenting cells, B cells that produce IgE, and mast cells, basophils, eosinophils, and other cells that bind IgE. These effector cells accumulate at the site of allergic reaction in the airways and release toxic products that contribute to the acute pathology and eventually to tissue destruction related to the disorder. Other resident cells, such as smooth muscle cells, lung epithelial cells, mucus-producing cells, and nerve cells may also be abnormal in individuals with asthma and may contribute to its pathology. While the airway obstruction of asthma, presenting clinically as an intermittent wheeze and shortness of breath, is generally the most pressing symptom of the disease requiring immediate treatment, the inflammation and tissue destruction associated with the disease can lead to irreversible changes that eventually make asthma a chronic and disabling disorder requiring long-term management.

Chronic obstructive pulmonary (or airways) disease (COPD) is a condition defined physiologically as airflow obstruction that generally results from a mixture of emphysema and peripheral airway obstruction due to chronic bronchitis [Botstein, 1980]. Emphysema is characterized by destruction of alveolar walls leading to abnormal enlargement of the air spaces of the lung. Chronic bronchitis is defined clinically as the presence of chronic productive cough for three months in each of two successive years. In COPD, airflow obstruction is usually progressive and is only partially reversible. By far the most important risk factor for development of COPD is cigarette smoking, although the disease does also occur in non-smokers.

GPCR is highly expressed in the following tissues of the respiratory system: leukocytes (peripheral blood), bone marrow CD15+ cells, lung, lung right upper lobe, lung right mid lobe, lung right lower lobe, lung tumor, lung COPD, trachea. The expression in the above mentioned tissues and in particular the differential expression between diseased tissue lung tumor and healthy tissue lung, between diseased tissue lung COPD and healthy tissue lung demonstrates that GPCR DNA, mRNA and polypeptides can be utilized to diagnose of respiratory diseases. Additionally the activity of GPCR can be modulated to treat those diseases.

Disorders Related to Urology

Genitourinary disorders comprise benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy, obstructive uropathies like benign prostatic hyperplasia (BPH), neurogenic bladder syndrome, urinary incontinence like urge-, stress-, or overflow incontinence, pelvic pain, and erectile dysfunction.

GPCR is expressed in the following urological tissues: ureter, penis, corpus cavernosum, fetal kidney, kidney, kidney tumor. The expression in the above mentioned tissues and in particular the differential expression between diseased tissue kidney tumor and healthy tissue kidney demonstrates that GPCR DNA, mRNA and polypeptides can be utilized to diagnose of urological disorders. Additionally the activity of the human GPCR can be modulated to treat urological disorders.

The present invention provides for both prophylactic and therapeutic methods for disorders of the peripheral and central nervous system, cardiovascular diseases, hematological diseases, cancer, inflammation, urological diseases, respiratory diseases and gastroenterological diseases.

The present invention provides methods of treating an individual afflicted with a disease or disorder characterized by unwanted expression or activity of GPCR or a protein in the GPCR signaling pathway. In one embodiment, the method involves administering an agent like any agent identified or identifiable in assays as described herein, or a combination of such agents to modulate expression or activity of GPCR or proteins in the GPCR signaling pathway. In another embodiment, the method involves administering a regulator of GPCR as therapy to compensate for reduced or undesirably low expression or activity of GPCR or a protein in the GPCR signaling pathway.

The expression in the above mentioned tissues and in particular the differential expression between diseased tissue spleen liver cirrhosis and healthy tissue spleen demonstrates that GPCR DNA, mRNA and polypeptides can be utilized to diagnose of hematological diseases. Additionally the activity of GPCR can be modulated to treat hematological disorders.

### EXAMPLES

The following examples are provided to illustrate the invention and are not intended to limit its scope. Other variants of the invention will be readily apparent to one of ordinary skill in the art and are encompassed by the appended claims.

### Example I

### Expression Vector Design

A vector (pMEX2) was designed to facilitate expression and detection of a GPCR expressed with correct orientation on the cytoplasmic membrane of mammalian cells. The vector contained a pUC origin and the beta-lactamase gene for replication and ampicillin selection of the plasmid in bacteria, as shown in FIG. 10. A puromycin resistance marker for maintaining the plasmid in mammalian cells is also included in the vector. Expression of the gene of interest was under control of a strong CMV promoter for high-level transcription activity. The expression cassette also contained a Kozak consensus sequence for optimal translation initiation and a SV40 late poly adenylation signal for stability of the transcripts.

Some features were added to the vector to facilitate isolation of GPCR cell lines. First, transportation of the receptor protein to the cytoplasmic membrane was greatly improved by fusion of an amino-terminal cleavable secretory signal peptide (amino acid seq: METDTLLLWVLLLWVPGSTGD, corresponding to SEQ ID NO. 19, position 1102 to position 1164) derived from a murine Ig kappa light chain. Second, a short affinity tag (Flag tag; amino acid seq: DYKDDDDK, corresponding to SEQ ID NO: 19, position 1168-1191) was fused downstream from the signal peptide and followed by a short flexible linker (glycine-serine-glycine) upstream from the mature sequence of the target gene.

Addition of the Flag tag at the amino-terminus of the receptor served two functions. First, it facilitated detection of the recombinant receptor and isolation of cells expressing the receptor. Secondly, it served as a marker for correct orientation of the receptor on the cytoplasmic membrane. Therefore, only receptors of correct orientation with the Flag tag exposed to the extracellular side of the cytoplasmic membrane can be detected by the mAb. The use of a universal affinity tag in this application is not limited to the Flag tag and can be extended to any sequence (e.g. Flag, myc, His tag, C9, HA etc.) which is recognized by a fluorescence-labeled binder, as long as it does not interfere with ligand binding to the fused receptor.

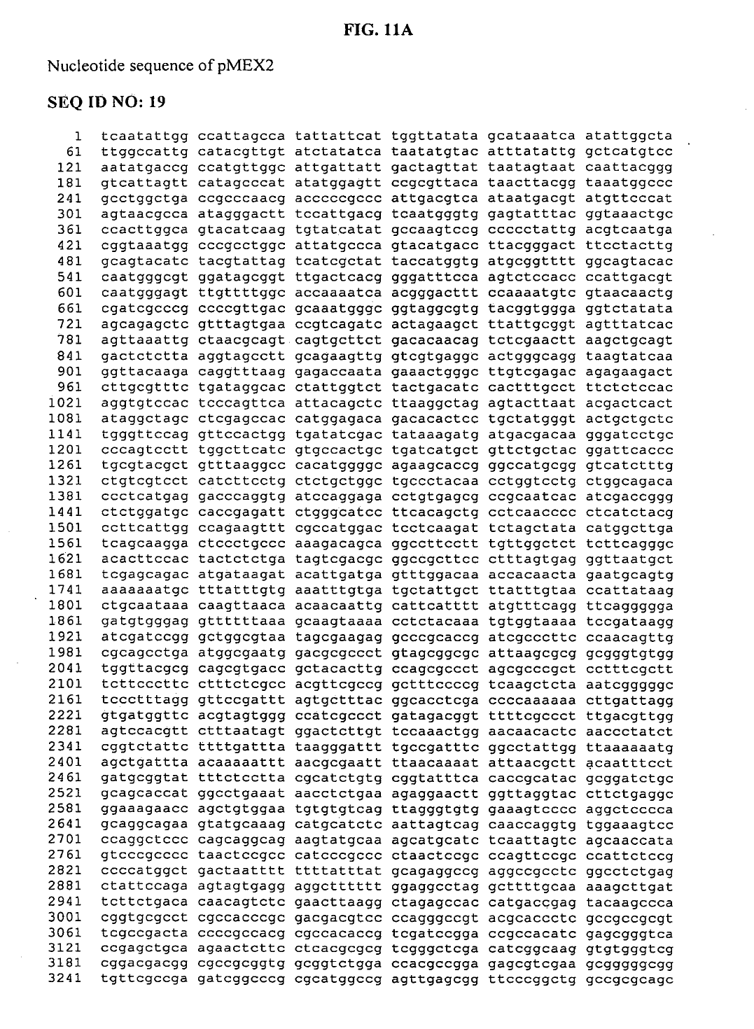

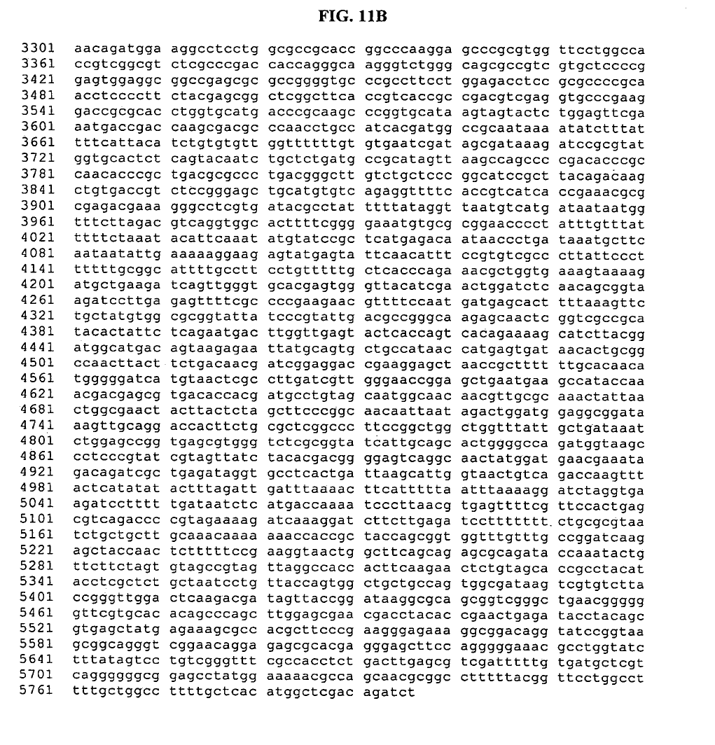

Upon translocation of the full-length protein to the membrane, the signal peptide was processed, leaving the receptor with an amino-terminal Flag tag exposed to the extracellular environment. Open reading frame of the GPCR to be expressed was amplified by polymerase chain reaction (PCR) from either a cDNA library or an EST clone and subcloned into the cloning sites (5′-BamH I and Sal I-3′) on the vector. The integrity of the gene sequence was confirmed by sequencing reactions through the whole insert using primers outside the cloning sites on the vector. A typical vector map of pMEX2 and its nucleotide sequence are shown in FIG. 10 and FIG. 11 respectively.

### Example II

### Screening for GPCR Clones

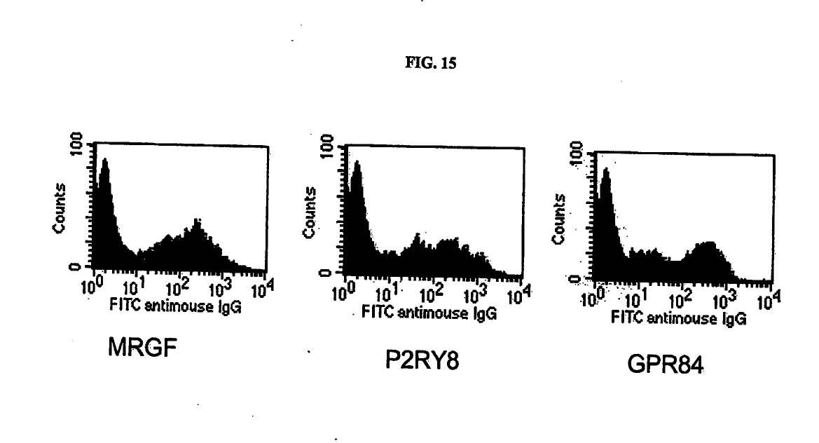

FACS analysis was carried out to confirm surface expression of the exogenously expressed receptors using the anti-Flag M2 mAb (Sigma) in a transient expression experiment. The plasmid DNA can be delivered into any mammalian cells using any method, including, but not limited to, electroporation, lipid cation and calcium phosphate-mediated transfection, or retrovirus-mediated infection. For the current experiments, the vector was transfected into either human embryonic kidney cells (HEK293) and its derivative (293T) or Chinese hamster ovary cells (CHO-K1) by Lipofectamine 2000 (Invitrogen) according to the manufacturer's protocol. Expression of the recombinant GPCR on cell surface was detected 48 to 60 hours post transfection by fluorescence-activated cell sorting (FACS). A typical expression profile for three GPCRs (MRGF, P2RY8 and GPR84) under transient expression condition is shown in FIG. 15 which shows broad spectrum of expression crossing two to three quadrants in the histograms.

### Example III

### Ligand-Binding Activity of the Recombinant Receptors

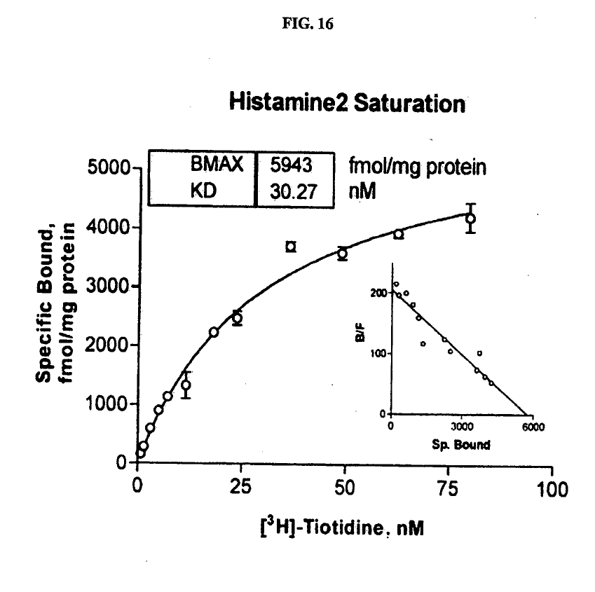

A saturation ligand-binding assay was performed to confirm that the recombinant receptors retain its native conformation and ligand-binding activity. The average ligand-binding sites (Bmax; pmol/mg protein) and the affinity (Kd; nM) of the exogenously expressed receptor in a transient expression experiment was measured at 12 radioligand concentrations with duplicate total and non-specific binding determinations. Representative result of specific binding for histamine receptor H2 is shown in FIG. 16. The human receptor exhibited similar affinity for the pig ligand as the endogenous receptor (data not shown). Based on the Bmax value and membrane protein expression per cell, the ligand binding sites was calculated to be 1.05 million copies/cell.

### Example IV

### Isolation of Stable Cell Lines Expressing High-Level GPCRs

Cell Culture.

HEK293 cells were maintained in DMEM supplemented with 10% FBS (Invitrogen Inc.), 100 U/ml penicillin/streptomycin. CHOK1 cells were maintained in either DMEM/Ham's F-12 Mix or Ham's F-12 media supplemented with 10% FBS 100 U/ml penicillin/streptomycin (Invitrogen Inc.). Cell transfections were carried out with Lipofectamine 2000 (Invitrogen Inc.) according to the manufacturer's protocol. In order to have large enough amounts of cells, one well of 6-well plate was at 80-90% confluence for HEK293T, Gα16/HEK293T, Gqi5/HEK293T, CHO-K1, Gα16/CHO-K1 and Gqi5/CHO-K1 cells or one T25 at 90% confluence for RH7777, 1321N1 cells.

Flow Cytometry Analysis.

To determine cell-surface GPCR expression, cells were detached from the plates with Cellstripper (Mediatech, Inc.). Cells were washed once with PBS, once with PBS/1% BSA at 4° C. They were incubated with mouse anti-Flag M2 mAb (Sigma Chemical Co) for 30 minutes at 4° C., washed twice with PBS/1% BSA at 4° C., and further incubated on ice in the dark with FITC-labeled goat anti-mouse IgG Sigma Chemical Co) as secondary antibody. Cells were washed twice again and resuspended in 400 μl PBS/1% BSA. The fluorescence of 10,000 cells/tube was assayed by a FACSort flow cytofluorometer (Becton Dickinson). For direct immunofluorescent staining, cells were first incubated with PE-labeled mouse anti-FLAG antibody (Prozyme Inc. San Leandro, Calif.) at 30 ug/ml for 30 minutes on ice, then washed and analyzed as described above.

Stable Pool Selection and Isolation of Stable Cell Lines Expressing High-Level GPCRs

To isolate stable populations, the cells were transfected in 6-well plates, transferred to 100-mm Petri dish two days post transfection. The stable pools for were selected by incubation of the cells in culture medium containing 1 ug/ml puromycin (InvivoGen Inc., San Diego, Calif.) for another 10 to 14 days for HEK293 transfectants, or medium containing 10 ug/ml puromycin for 7 to 10 days for CHOK1 transfectants.

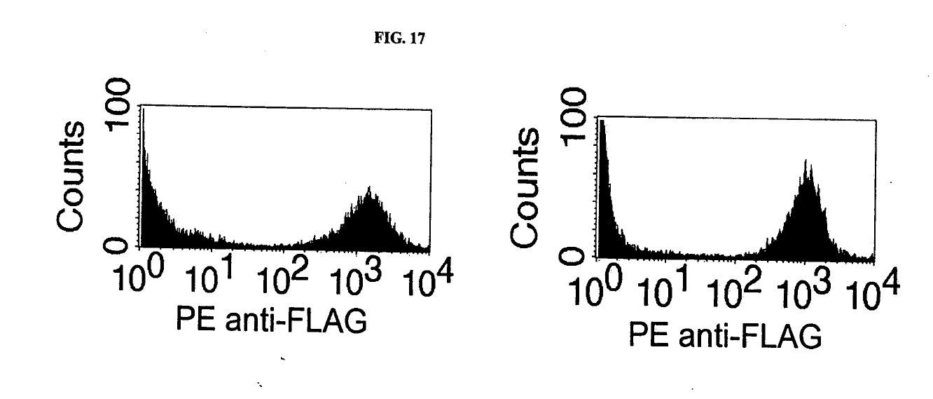

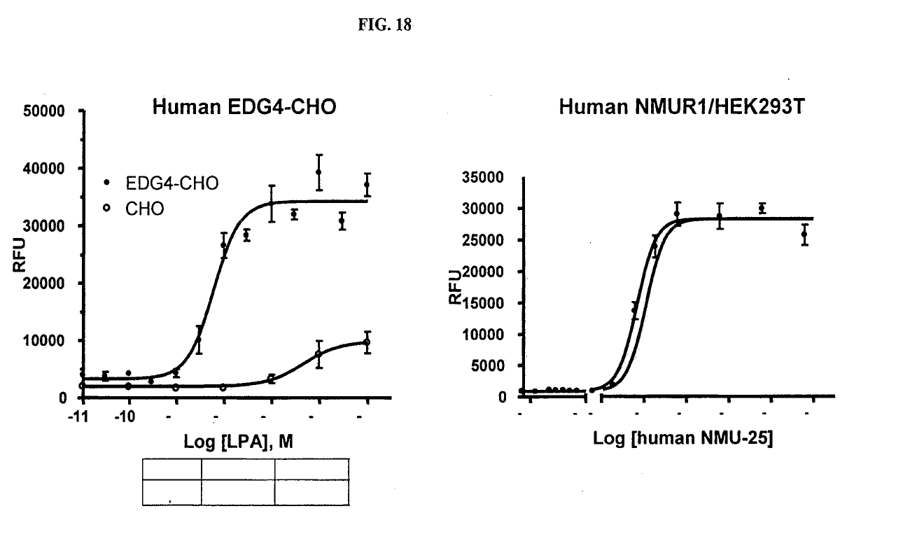

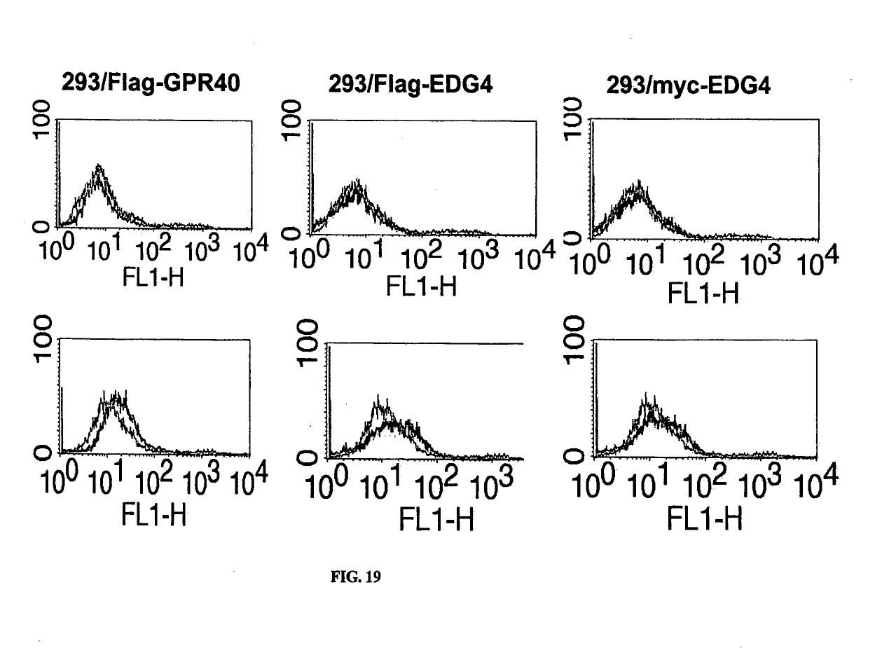

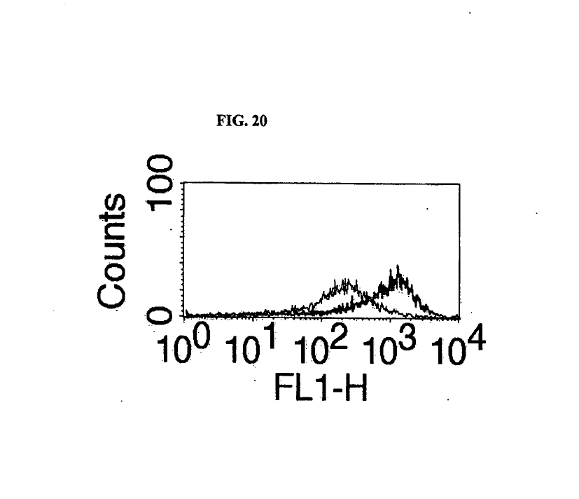

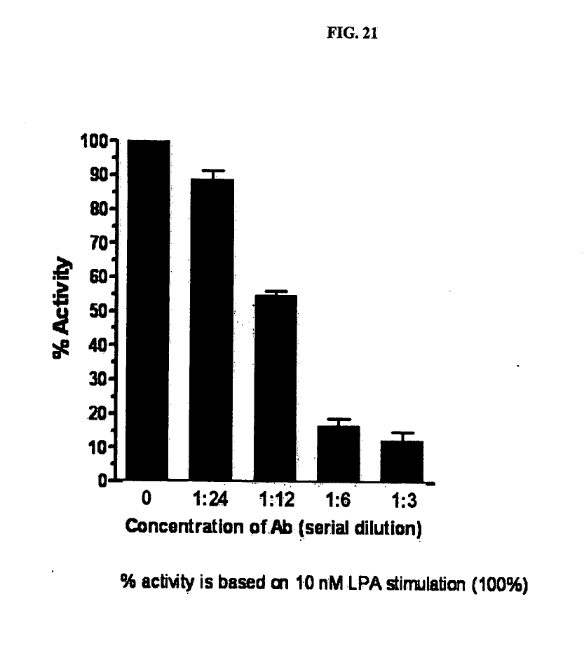

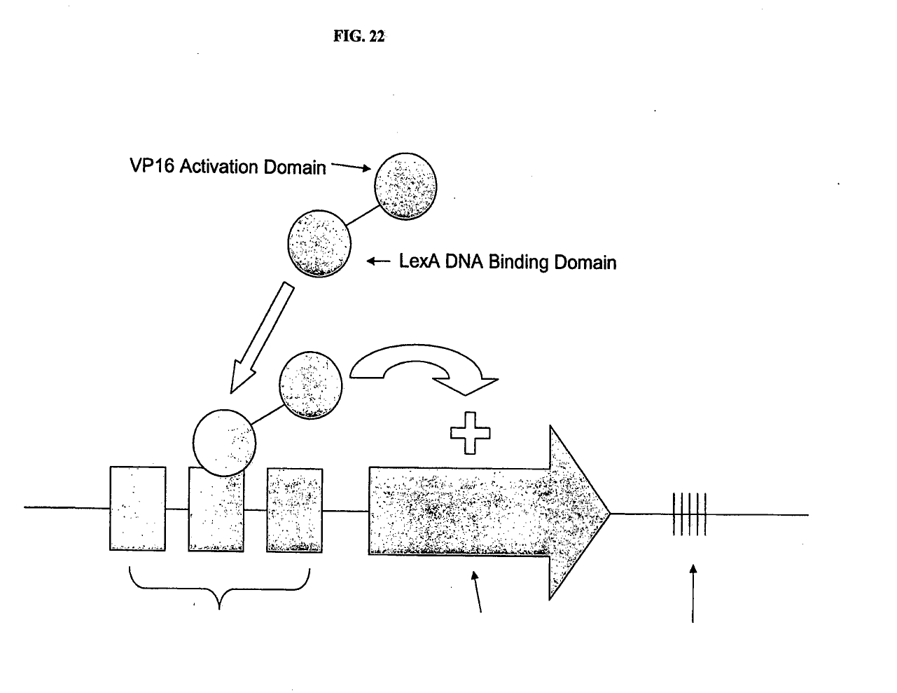

To isolate single cells expressing high levels of the receptors, the stable colonies were pooled together, subjected to cell sorting with FACSort (Becton Dickinson) to separate the high expressers from the rest of the population. The top 0.5% to top 5% high expressers in the total stable population were gated and sorted out subsequently into sterile 50-ml conical tubes pre-soaked with 4% BSA in PBS. The cells were concentrated by centrifugation and either plated on 96-well plates by serial dilution at 0.5, 1, and 5 cells/well, or plated in 150-mm Petri dish in medium containing puromycin. Single-cell colonies were confirmed by examination under a microscope, expanded into 24-well plates, and analyzed by FACS analysis. One such recombinant CHO clone, 9-4, which expresses high level of the human lysophosphatidic acid receptor 4 (EDG4) in CHO-K1 cells is shown by FACS analysis in FIG. 18. The FACS profile for the recombinant HEK293 cell for NMUR1 (colony 3) is shown in FIG. 17. The expression level of EDG4 in CHOK1 (clone 9-4) and NUMR1 in HEK293 (colony 3) are above 200,000 copies per cell, as measured using calibrated standardized phycoerythrin (PE)-conjugated beads (BD QuantiBRITE) and PE-conjugated M2 antibody.

The high-expressing cell lines exhibited good cellular response (such as intracellular calcium release) upon ligand binding to the receptors. The developed GPCR expressing cell lines provide critical tools for cell-based functional drug screening, in vitro ligand-binding assays, crystallization for structural studies and the development of monoclonal antibodies with the use of the recombinant whole cells as immunogens. Data for two representative cell lines are shown in FIG. 17.

### Example V

### Antibody Development: Whole Cell Immunization Strategy

Immunization of Animals.

GPCR expressing isogenic cell line at an expression level of >100,000 copies per cell was used as immunogen. Female Balb/c mice of 6-8 weeks old are primary choice of animal. Each mouse is immunized with 5-10 millions of live cells per mouse. Mouse: 6-8 weeks Balb/c mouse as primary strain. Another strain with different MHC II type as backup: Live cells (about 10⁶ per mouse) plus Freund's adjuvant. Cells are destroyed during emulsifying antigen.

On the first day, pre-immune bleeds are taken. Cells (about 20 million) are harvested and washed 3× with large volume (40 ml) of PBS, and re-suspended in PBS. Cell suspension is mixed with equal amount of Complete Freund's adjuvant (CFA), emulsified and injected about 5 millions of cells per mouse intraperitoneally.

The Day 1 procedure was repeated over the next few weeks (except no adjuvant every other 2 weeks) until a specific antibody titer of >1:50,000 is reached.

Tail bleeds were taken 7-10 days after the third, sixth and ninth immunizations and will be tested by whole cell ELISA against immunogen-expressing and non-expressing cells. The cells used in whole cell ELISA ideally should be different from immunogen. Best responders (antibody titer >1:50,000) against desired antigen is expected.

Final boost was given to the best responder(s), 2.5 millions of cells iv and 2.5 millions of cells ip 3-4 days before the hybridoma fusion.

Titering of Sera.

Serum titers for individual mice within each immunization group were determined in duplicate against the corresponding screening antigens (both cells expressing and non-expressing desired GPCR). Groups of mice that failed to show titers after extended periods of immunization will be terminated.

Lymphoid Organ Harvest.

Immunoresponsive mice as determined by titering were used for hybridoma generation and the designated lymphoid organs (spleen in one embodiment) harvested and processed using conventional practices for B cell isolation.

Fusion.

The fusion was done the same day following a standard hybridoma fusion protocol. SP2/0 mouse myeloma cell line will be used as fusion partner. The plated fusion products (10-20 plates per fusion) were plated at intermediate density (not more than 10̂7 cells per plate), and then cultured for 11-14 days prior to screening.

Primary Screening.

Cell culture supernatants were tested against the relevant screening antigens with a whole cell ELISA protocol. Anti-mouse IgG antibody-enzyme reporter conjugate were used to detect antigen specific immunoreactivity in the wells and identify those of interest for retesting and potential cloning. Following data analysis, all strong positive lineages were picked into 24-well cell culture plates.

Cell Expansion.

The cells in 24-well cell culture plates were cultured to exhaustion and cell culture supernatants (˜2 ml) were recovered to verify the original screening antigen reactivities.

Secondary Screening.

All 24-well cell culture supernatants were re-tested against the relevant screening antigens following the protocol in Appendix 3 and with the desired applications, such as, Western Blot, ImmunoPrecipitation, Flow Cytometry and immunohistochemistry. Cell lines were also banked at this stage. This activity at times required up to 14 days. Following data analysis, line supernatants whose screening antigen reactivity were verified were advanced to subcloning.

Subcloning.

Subcloning by limiting dilution for all hybridoma lines selected were conducted. If there were multiple clones that showed exactly the same functionalities, only the two best were cloned. Up to 3 daughter clones were selected from each linage, based on visual inspection, production and immunoreactivity. Clones were screened with the relevant screening antigen approximately 10-12 days post subcloning, depending on individual growth rates in culture. The complete subclonings and screening procedures required up to 4 weeks, depending how many rounds of subclonings were needed.

The final clones from previous work were processed to scaling up in this phase through ascites generation. Antibody purification was needed depending on the result of application.

### Example VI

Hybridoma Screening

ELISA plates (Corning 3369 or similar) were coated with 100 μl of high-density (concentration to be decided) cells expressing desired proteins (in PBS). The plates were allowed to air-dry inside a cell culture hood at room temperature for overnight. Negative control cells (non-transfected cells) were processed in parallel with the transfected cells.

After overnight culture, the plates were washed three times with PBS+0.05% Tween-20 (PBST) and then blocked with 250 μl/well of PBST-5% skim milk). The plates were then incubated at room temperature for 1 hour (or at 4° C. overnight).

After incubation, the PBST-5% skim Milk was discarded and 50 μl/well cell culture supernatant or other form of testing antibodies was added, followed by another incubation for one hour at room temperature (or overnight at 4° C.).

After another 3× wash with PBST, 1:10,000 diluted goat anti-mouse IgG-HRP conjugate (Jackson Immuno 115-036-071 or similar) was added the plates incubated at room temperature for another hour.

After the incubation and a five time wash in PBST, HRP (horseradish peroxidase) substrate, Sigma Fast OPD was added and the plates incubated in the dark at room temperature for 30-60 min.

Plates were read at OD450 with a 96-well colormetric detector if reaction was not stopped, or at OD492 if stopped with 1.25M sulfuric acid.

### Example VII

### FACS, Sorting Protocol for Establishing Stable Cell Line

This protocol was designed for use in conjunction with an anti-flag-tag antibody (clone M2 from Sigma-Aldrich) and the mammalian cells transfected with pMEX plasmids carrying a full-length GPCR gene. A fraction of cells, 100K cells, were used to determine GPCR expression level by FACS on FACSort (Becton Dickinson) following a 96-well-microtiter-plate (use any U-shape plate) protocol below:
1. Resuspend cells and transfer them to a 15-ml centrifuge tube, 1200 rpm 5 min;
2. Wash cells once with 10 ml of cold PBS;
3. Wash cells with 10 ml of cold PBS+1% BSA (FB);
4. Resuspend the cells with cold FB and add 100 μl per well;
5. 1200 rpm 2 min, and flick the plate;
6. Vortex briefly to suspend the cells;
7. Add 100 μl of M2 antibody (anti-flag-tag) at 10 μg/ml;
8. Put the plate on top of ice for 30 min;
9. Spin-flick to remove M2 antibody;
10. Wash 2× with 250 μl FB;
11. Vortex, add to each well 100 μl of FITC-labeled antibodies (anti-mouse) at about 10 per ml (1:100 dilution from a 1 mg/ml product);
12. Put the plate on top of ice for 20 min;
13. Spin-flick, then Wash 2× with 250 μl FB;
14. Resuspend cells in 250 μl FB and transfer cells to FACS tubes;
15. Ready for FACS.
   Two to three cells were collected. The collected cells were centrifuged and resuspended in selection medium, and immediately aliquoted 100 μl or one cell per well to 96-well plate. Cell number was estimated based on the number of cells collected during the sorting.

### Example VIII

### Protocol for Calcium Mobilization Assay

The Gq-coupled GPCRs expressed in CHO or HEK293T or other cells and the GPCRs coupled with other G-proteins were expressed in the cells transfected with chimeric G-proteins. Functional expression was tested using FLIPR Calcium 4 Assay Kit (Molecular Devices) using the standard protocol, which is reproduced as follows:

A. Preparation of Cells
1. Culture adherent cells in 96-well ploy-D-lysine-coated microplates (Sigma, cat# M-5307), to near confluence. CHO cells can be plated at 30,000-40,000 cells per well and grown overnight. HEK293 cells can be plated at 40,000-50,000 cells per well and grown overnight.

B. Preparation of Reagents
2. Make a 250 mM stock of probenecid acid (100×): dissolve in 1 N NaOH and neutralize with equal volume of HBSS/HEPES.
3. Prepare the dye loading solution (for one microplate): add 10 ml of assay buffer and 100 of probenecid acid (final concentration: 2.5 mM) stock solution to a vial of dye mix. Vortex for 1 minute to ensure a complete dissolving.
4. Prepare a solution of receptor agonist (3×) in assay buffer with 0.1% BSA. Make serial dilution in 96-well compound plate (VWR #62409-112, NUNC, V-bottom)

C. Assay
5. Remove the growth medium from the adherent cell cultures. Quickly but carefully add 100 μl of the dye loading solution to each well of a 96-well plate.
6. Incubate the plate at 37° C. for 1 hour.
7. Measure fluorescence using Flexstation (Molecular Device). Instrument settings: excitation at 485 nM, emission at 525 nM, cut-off at 515 nM, compound addition (transfer volume): 50 μl, addition speed (rate): 2, pipette height: 80 μl, assay duration 2-3 minutes.
HBSS/HEPES: Hanks' Balanced Salt Solution (1×) with 20 mM HEPES, pH 7.4.

Although the invention has been described with reference to the presently preferred embodiments, it should be understood that various modifications can be made without departing from the spirit of the invention. Accordingly, the invention is limited only by the following claims.

What is claimed is:

1. A method of screening for therapeutic candidates, comprising:
detecting the number of GPCRs expressed on the surface of an isolated recombinant cell expressing a GPCR protein;
contacting the cell with a test entity;
detecting the number of GPCRs on the cell surface after contact with the test entity; and
determining the difference between the numbers;
wherein:
an affinity tag is attached to the GPCR protein that allows for detection of the GPCRs on the cell surface; and
a decrease in the number of GPCRs on the cell surface after contact with the test entity identifies the test entity as a therapeutic candidate.

2. The method of claim 1, wherein the expression of the GPCR protein is governed by a high expression vector that facilitates expression of at least about 150,000 copies of the GPCR protein prior to contact with the test entity.

3. The method of claim 2, wherein the recombinant cell expresses from about 150,000 to about 2,000,000 copies of the GPCR protein prior to contact with the test entity.

4. The method of claim 2, wherein the expression vector has a nucleotide sequence selected from SEQ ID NO: 19 and SEQ ID NO: 20.

5. The method of claim 1, wherein the affinity tag is attached to the amino-terminal of the GPCR protein and exposed to the extracellular environment.

6. The method of claim 1, wherein the amino acid sequence of the affinity tag comprises Asp-Tyr-Lys-Asp-Asp-Asp-Asp-Lys.

7. The method of claim 6, wherein the detecting comprises contacting the affinity tag with an antibody or antigen binding fragment that binds to the affinity tag.

8. The method of claim 7, wherein the antibody or antigen binding fragment is monoclonal.

9. The method of claim 1, wherein determining the number of GPCRs on the surface of the cell is performed by a high throughput screen.

10. The method of claim 1, wherein determining the number of GPCRs on the surface of the cell is performed by flow cytometry or fluorescence-activated cell sorting.

11. The method of claim 1, wherein the GPCR protein is a member of a GPCR family selected from: an anaphylatoxin receptor, an apelin receptor, a bombesin receptor, a cannabinoid receptor, a chemokine receptor, a free fatty acid receptor, a galanin receptor, a glucagon receptor, a glycoprotein hormone receptor, a leukotriene/lipoxin receptor, a lysophospholipid receptor, a melanin-concentrating hormone receptor, a melatonin receptor, a N-formylpeptide receptor, a neuromedin U receptor, a neuropeptide S receptor, a neuropeptide W/neuropeptide B receptor, a neuropeptide Y receptor, an opioid receptor, a platelet activating factor receptor, a prolactin releasing peptide receptor, a prostanoid receptor, a PTH receptor, a purinergic receptor, a tachykinin receptor, a trace amine receptor, and a urotensin receptor.

12. The method of claim 1, wherein the GPCR protein is an orphan GPCR.

13. The method of claim 1, wherein the GPCR protein is selected from: C3aR, APJ, BB1, BB3, GPR55, CCR1, CCR5, CCR7, CCR9, CMKLR1, CXCR3, CXCR4, FFA1, FFA2, GAL1, GAL2, GAL3, GHRH, TSH, ALX, BLT1, BLT2, CysLT1, LPA2, LPA3, MCH1, MT2, FPR1, NMU1, NPS, NPS(1), NPS(2), NPS Ile107, NPBW1, NPBW2, delta, kappa, mu, NOP, GPR37L1, GPR84, MRGX1, MRGX2, PSGR, PAF, PRP, DP, EP1, GPR44, PTH2, P2Y12, NK2, NK3, TA1, C5AR, and PAR2.

14. A method of screening drug candidates, comprising:
a) culturing a cell line comprising:
an isolated recombinant cell comprising a high expression vector that facilitates expression of at least 150,000 copies of a GPCR protein on the cytoplasmic membrane of a cell;
in vitro under conditions suitable for expression of the GPCR protein;
b) using flow cytometry to detect the number of GPCRs expressed on the surface of the cells of the cell line;
c) contacting the cells with a drug candidate;
d) using flow cytometry to detect the number of GPCRs on surface of the cells after contact with the drug candidate; and
e) determining the difference between the numbers;
wherein:
the expression vector attaches an affinity tag to the GPCR protein that allows for detection of the GPCRs on the cell surface.

15. The method of claim 14, wherein the number of GPCRs decreases after contact with the drug candidate.

16. The method of claim 14, wherein the drug candidate modulates the activity of the GPCR protein.

17. The method of claim 14, wherein the affinity tag is attached to the amino-terminal of the GPCR protein and exposed to the extracellular environment, and the amino acid sequence of the affinity tag comprises Asp-Tyr-Lys-Asp-Asp-Asp-Asp-Lys.

18. The method of claim 14, wherein the GPCR protein is a member of a GPCR family selected from: an anaphylatoxin receptor, an apelin receptor, a bombesin receptor, a cannabinoid receptor, a chemokine receptor, a free fatty acid receptor, a galanin receptor, a glucagon receptor, a glycoprotein hormone receptor, a leukotriene/lipoxin receptor, a lysophospholipid receptor, a melanin-concentrating hormone receptor, a melatonin receptor, a N-formylpeptide receptor, a neuromedin U receptor, a neuropeptide S receptor, a neuropeptide W/neuropeptide B receptor, a neuropeptide Y receptor, an opioid receptor, a platelet activating factor receptor, a prolactin releasing peptide receptor, a prostanoid receptor, a PTH receptor, a purinergic receptor, a tachykinin receptor, a trace amine receptor, and a urotensin receptor.

19. The method of claim 14, wherein the GPCR protein is an orphan GPCR.

20. The method of claim 14, wherein the GPCR protein is selected from: C3aR, APJ, BB1, BB3, GPR55, CCR1, CCR5, CCR7, CCR9, CMKLR1, CXCR3, CXCR4, FFA1, FFA2, GAL1, GAL2, GAL3, GHRH, TSH, ALX, BLT1, BLT2, CysLT1, LPA2, LPA3, MCH1, MT2, FPR1, NMU1, NPS, NPS(1), NPS(2), NPS Ile107, NPBW1, NPBW2, delta, kappa, mu, NOP, GPR37L1, GPR84, MRGX1, MRGX2, PSGR, PAF, PRP, DP, EP1, GPR44, PTH2, P2Y12, NK2, NK3, TA1, C5AR, and PAR2.
